(19) United States
(12) Patent Application Publication
(10) Pub. No.: US 2011/0189291 A1
(43) Pub. Date: Aug. 4, 2011

(54) DENDRIMER HYDROGELS

(76) Inventors: Hu Yang, Mechanicsville, VA (US); Pooja N. Desai, Glen Allen, VA (US); Christopher A. Holden, Broad Run, VA (US); Uday B. Kompella, Englewood, CO (US); Puneet Tyagi, Denver, CO (US)

(21) Appl. No.: 13/022,974

(22) Filed: Feb. 8, 2011

Related U.S. Application Data
(63) Continuation-in-part of application No. PCT/US09/52678, filed on Aug. 4, 2009.

(51) Int. Cl. A61K 9/00; A61K 31/5377; A61P 27/02

(52) U.S. Cl. 424/486; 514/236.2

(57) ABSTRACT

Photoactivatable dendrimers and hydrogels formed therefrom include dendrimers to which polymer chains (e.g. polyethylene glycol, PEG) have been conjugated; and reactive photoactivatable groups attached to terminal functional groups of the polymer chains (e.g. hydroxyls of PEG). Exposure to a suitable wavelength of light activates the photoactivatable groups, which then crosslink with one another, thereby forming a hydrogel. The hydrogel may also include one or more agents of interest; or, in some embodiments, nanoparticles containing one or more agents of interest may be dispersed in the hydrogel. These formulations are well-suited for sustained or prolonged delivery of active agents, e.g. for the treatment of glaucoma by the sustained delivery of anti-glaucoma agents directly to the eye.

This application claims benefit of and is a continuation-in-part of International patent application PCT/US2009052678, filed Aug. 4, 2009, and U.S. provisional patent application 61/087,209 filed Aug. 8, 2008, the complete contents of both of which are hereby incorporated by reference.

### BACKGROUND OF THE INVENTION

1. Field of the Invention

The invention generally relates to photoactivatable dendrimers and hydrogels formed therefrom. In particular, the invention provides dendrimers with multiple conjugated polymer chains (e.g. multiple polyethylene glycol, PEG, chains) and photoactivatable reactive groups attached to the terminal end of the conjugated polymer chains. Exposure to suitable wavelengths of light causes crosslinking of the reactive groups, and hence the formation of a hydrogel. In a formulation that is especially suited for extended delivery, the hydrogel further comprises one or more agents of interest; or, in some embodiments, nanoparticles containing one or more agents of interest are dispersed in the hydrogel.

2. Background of the Invention

Hydrogels are crosslinked insoluble networks of polymer chains that swell in aqueous solutions, and which have found many applications including drug delivery and tissue regeneration. Hydrogels are useful in biomedical and pharmaceutical applications because of their biocompatibility, high water content, low surface tension, hydrodynamic properties that are very similar to those of natural biological gels and tissues, and their minimal mechanical irritation due to their soft and rubbery state. Due to their high water content, these gels resemble natural living tissue more than any other type of synthetic biomaterial. In addition to being used as carriers of bioactive agents, they can also provide protection for proteins or drugs. The perm selective nature of hydrogels makes them suited for diverse applications ranging from controlled drug delivery to cellular and tissue transplantation.

Dendrimers provide an ideal platform for drug delivery as they possess a well-defined highly branched nanoscale architecture with many reactive surface groups. Drug molecules either can be physically entrapped inside the dendritic structure or can be covalently attached onto the surface. In particular, their highly clustered surface groups allow for targeted drug delivery and high drug payload to enhance therapeutic effectiveness. Dendrimers have also been studied as crosslinking agents because of their multiple reactive surface groups. In particular, hydrogels formulated based on PEGylated dendrimers are of great interest because they have many biologically favorable properties. For example, they have found applications in cartilage tissue formation, and for sealing ophthalmic wounds. These hydrogels prove effective due to the presence of dendritic macromolecules which are highly branched and which possess multiple sites having many reactive end groups which enable appropriate crosslinking and impart multiple hydrogel properties. The surface charges conferred by terminal groups on the dendrimer surface can make the hydrogel polyionic with controllable charge density.

Hydrogels can be classified into ionic, non ionic, and neutral hydrogels. Ionic hydrogels have the ability to respond to changes of pH, hence termed as pH sensitive hydrogel. Ionic hydrogels have two main structure features: a penetrable network and a number of fixed charges. The penetrable network allows the exchange of solute and water. Fixed charges are responsible for the regulation of the electrochemical balance between the hydrogel and the surrounding medium. The swelling of ionic hydrogels is governed by pH. For instance, a hydrogel network containing acidic groups swells at high pHs but shrinks at low pHs. Therefore ionic hydrogels have been used for delivery of various therapeutics and controlled drug release based on pH adjustment.

Due to these and many other potential applications, there is an ongoing need to develop improved dendrimers and dendritic hydrogels with increasingly flexible architectures, and which are capable of being adapted to a variety of uses and conditions.

### SUMMARY OF THE INVENTION

The present invention provides photoactivatable dendrimers and hydrogels formed from photoactivated dendrimers. Each photoactivatable dendrimer is comprised of a dendrimer to which a plurality of polymer chains have been conjugated, and reactive photoactivatable groups attached to terminal functional groups of the conjugated chains. Upon exposure to a suitable wavelength of light, the photoactivatable groups become crosslinked to one another, thereby covalently linking adjacent dendrimers to each other and forming a hydrogel. In one embodiment, the photoactivatable dendrimer is a PEGylated dendrimer (i.e. a dendrimer to which multiple polyethylene glycol, PEG, chains of varying lengths have been conjugated); and the reactive photoactivatable groups are attached to the terminal hydroxyl groups of the PEG chains. The hydrogel may further comprise one or more agents of interest (e.g. drugs or therapeutic agents, especially those for which slow or sustained release is desirable); or, in some embodiments, the one or more agents of interest may be incorporated into or contained within nanoparticles that are dispersed in the hydrogel. In yet other embodiments, both the hydrogel itself and nanoparticles dispersed in the hydrogel may contain one or more active agents of choice. The hydrogels of the invention are particularly well suited for long-term, sustained delivery of active agents.

It is an object of the invention to provide hydrogel-nanoparticle dispersions. The dispersion comprises i. a hydrogel; and, ii. nanoparticles dispersed in the hydrogel. The hydrogel comprises a plurality of dendrimers, and a plurality of crosslinked conjugated polymer chains. The polymer chains are conjugated to the dendrimers, typically at least 3-4 polymer chains to each dendrimer. The plurality of dendrimers are connected to one another and form a “network” via the crosslinking of the polymer chains. The conjugated polymer chains are crosslinked at their termini. In one embodiment of the invention, the dendrimers are polyamidoamine (PAMAM) dendrimers, for example, PAMAM G3.0 dendrimers. In another embodiment, the conjugated polymer chains are polyethylene glycol (PEG) chains. In some embodiments, the PEG chains have a molecular weight of 12,000 Da. In other embodiments of the invention, the nanoparticles are formed from copolymers of lactic acid and glycolic acid (PLGA), for example, PLGA with a molecular weight of, for example, about 2,000 to about 100,000. In some embodiments, the PLGA Mr is in the range of form about 30,000 to about 35,000 Da. In some embodiments of the invention, the mass ratio of PLGA to hydrogel is 1:16.2.

In yet other embodiments, the nanoparticles comprise at least one medicament, for example, at least one medicament that is a drug for treating a disease of the eye. In another embodiment, the disease of the eye is glaucoma and the at least one medicament is one or both of timolol and brimonidine, or suitable salts thereof, e.g. timolol maleate. In this embodiment, the at least one medicament includes 3.5% weight of timolol maleate per volume of hydrogel-nanoparticle dispersion and 0.7% weight of brimonidine per volume of hydrogel-nanoparticle dispersion. In another embodiment, the nanoparticles are formed from PLGA and a weight ratio of timolol maleate to PLGA in the nanoparticles is 40:100 and a weight ratio of brimonidine to PLGA is 20:100.

The invention further provides a method for treating glaucoma in an eye of a patient in need thereof. The method comprises the step of administering to the eye of the patient a hydrogel-nanoparticle dispersion, comprising a dendrimer hydrogel and nanoparticles dispersed in the hydrogel, the nanoparticles containing or loaded with at least one anti-glaucome agent, e.g. one or both of timolol and brimonidine, or suitable salts thereof such as timolol maleate. The hydrogel comprises a plurality of dendrimers, and a plurality of crosslinked conjugated polymer chains. The polymer chains are conjugated to the dendrimers, typically at least 3-4 polymer chains to each dendrimer. The plurality of dendrimers are connected to one another and form a “network” via the crosslinking of the polymer chains. The conjugated polymer chains are crosslinked at their termini. In one embodiment of the invention, the dendrimers are polyamidoamine (PAMAM) dendrimers, for example, PAMAM G3.0 dendrimers. In another embodiment, the conjugated polymer chains are polyethylene glycol (PEG) chains. In some embodiments, the PEG chains have a molecular weight of 12,000 Da. In other embodiments of the invention, the nanoparticles are formed from copolymers of lactic acid and glycolic acid (PLGA), for example, PLGA with a molecular weight of e.g. about 2,000 to about 100,000, or in some embodiments, about 30,000 to 35,000 Da. In some embodiments of the invention, the mass ratio of PLGA to hydrogel is 1:16.2. In some embodiments, timolol maleate is present at 3.5% weight per volume of hydrogel-nanoparticle dispersion and brimonidine is present at 0.7% weight per volume of hydrogel-nanoparticle dispersion. In other embodiments, a weight ratio of timolol maleate to PLGA is 40:100 and a weight ratio of brimonidine to PLGA is 20:100.

The invention also provides a method for forming a dendrimer hydrogel. The method comprises the steps of 1) covalently attaching photoactivatable reactive groups to terminal diol moieties of a plurality of polyethylene glycol (PEG)-diol polymer chains, thereby forming photoactivatable PEG polymer chains; 2) attaching the photoactivatable PEG polymer chains to a plurality of dendrimers; and 3) exposing a plurality of dendrimers with attached photoactivatable PEG polymer chains to a wavelength of light that causes cross-linking between photoactivatable reactive groups of the photoactivatable PEG polymer chains. This results in linking the plurality of dendrimers to each other via the crosslinked PEG polymer chains, and the formation of a dendrimer hydrogel. In some embodiments, nanoparticles are dispersed within the dendrimer hydrogel.

### BRIEF DESCRIPTION OF THE DRAWINGS

FIG. 1A-C. Schematic depiction of A, a photoactivatable dendrimer and B, a hydrogel formed from crosslinked photoactivated dendrimers; C, hydrogel with dispersed nanoparticles.

FIGS. 2A and B. A, Conjugation of PEG to the dendrimer. The feeding molar ratio of OH-PEG-NPC/dendrimer was reduced to 4:1 to prepare a lower degree of PEGylation on the dendrimer surface following the same procedure as described in Example 1. B, Chemistry for introduction of a UV sensitive double bond to PEGylated G3.0.

FIG. 3. Comparison of water swelling study of interpenetrating network (IPN) composed of low PEGylated dendrimer G3.0 hydrogel and G3.5 dendrimer-PEG (1500) after 24 hours of incubation.

FIG. 4. Photograph of hydrogel formed on a polytetrafluoroethylene (PTFE) substrate.

FIG. 5. Release of cyclosporine A from half generation based dendrimer (G3.5-[PEG 1500-acrylate] 43) hydrogel at different pHs in 100 ml of medium.

FIG. 6. A highly adaptable and multifunctional polyamidoamine (PAMAM) dendrimer platform for ocular drug delivery. Dendritic cores are able to encapsulate hydrophobic drugs, the dendritic surface allows for covalent drug conjugation and assembly of various functional moieties, and the cross-linked PEG network delivers hydrophilic drugs. In addition, PAMAM dendrimers surface confers numerous positive charges, making the hydrogel have superior tissue adhesiveness.

FIG. 7. MTT assay used to judge the effect of hydrogel formulations on HCET cells. HCET cells were incubated with 300 each of four hydrogel formulation and incubated for 24 hours. MTT reagent was added after incubation. Absorbance after treatment with MTT reagent was measured by UV spectrophotometer. Data is presented as mean±SD. * indicates P<0.05 compared to blank.

FIG. 8. Protein content estimated for the cells after MTT assay had been performed. Micro BCA® Protein Assay Kit was used to estimate the protein content. Each of the well contents (150 μl) was added to 150 μl of the reagent mixture and kept at 37° C. for 2 hours. Data is presented as mean±SD.

FIG. 9. MTT assay and protein assay results of Formulation C in FIG. 7 and FIG. 8 were normalized to control and reported for n=3.

FIG. 10. In vitro release of brimonidine and timolol maleate. 100 μl of drug loaded hydrogel formulation and eye drop formulation was transferred to dialysis membrane and suspended in the dissolution media. Entire dissolution media was replaced at specific time intervals and amount of drug released estimated using LC-MS/MS. Data is presented as mean±SD for n=3.

FIGS. 11A and B. Cumulative percentage transport of hydrogel and solution, both containing 0.1% brimonidine (A) and 0.5% timolol maleate (B) across bovine cornea. For brimonidine, statistically significant differences (p<0.05) were observed in transcorneal transport from hydrogel and solution starting from 3 h. For timolol maleate, statistically significant differences (p<0.001) were observed in transcorneal transport from hydrogel and solution starting from 2 h.

FIG. 12A-F. Bovine corneal tissue (epithelium, stroma and endothelium) uptake of brimonidine (A-C) and timolol maleate (D-F) from their hydrogel and solution dosage forms after 1 h of topical instillation. Hydrogel formulation contained 0.1% brimonidine and 0.5% timolol maleate. Plain solution also contained 0.1% brimonidine and 0.5% timolol maleate. For each formulation, 4 eyes were used. Levels of timolol maleate were significantly higher (p<0.05) from the hydrogel than solution in epithelium, stroma and endothelium.

FIGS. 13A and B. Bovine aqueous humor uptake of brimonidine (A) and timolol maleate (B) from their hydrogel and solution dosage forms after 1 h of topical instillation. Hydrogel formulation contained 0.1% brimonidine and 0.5% timolol maleate. Plain solution also contained 0.1% brimonidine and 0.5% timolol maleate. For each formulation, 4 eyes were used.

FIG. 14. Intraocular pressure measurements observed in Dutch belted rabbits after topical administration of hydrogel formulation. Data is expressed as mean±S.D. for n=3.

FIG. 15. Intraocular pressure measurements observed in Dutch belted rabbits after topical administration of nanoparticle formulation. Data is expressed as mean±S.D. for n=3.

FIG. 16. Intraocular pressure measurements observed in Dutch belted rabbits after topical administration of PBS dispersion formulation. Data is expressed as mean±S.D. for n=3.

FIG. 17A-C. Graph showing nanoparticle content (% of nanoparticle dose) in different solutions after incubation for 5 minutes (A), 60 minutes (B), and 3 hours (C). HCET cells were to plated in a 48 well plate (surface area 0.95 cm²). At 80% confluency, Nile red loaded nanoparticles entrapped in hydrogel or dispersed in phosphate buffer saline (PBS) were added to the wells. After the incubation (5 minute, 60 minute, and 3 hours), cells were lysed using 2% Triton X 100 solution in PBS. Nile red fluorescence in the nanoparticles present in the cell lysate was measured spectrophotometrically. Data is shown as mean (±S.D.) for n=6.

FIG. 18. Nanoparticle content (% of nanoparticle dose) observed in cell lysate after incubation time of 5 minute, 60 minute, and 3 hours. HCET cells were plated in a 48 well plate (surface area 0.95 cm²). At 80% confluency, Nile red loaded nanoparticles entrapped in hydrogel or dispersed in phosphate buffer saline (PBS) were added to the wells. After the incubation (5 minute, 60 minute, and 3 hours), cells were lysed using 2% Triton X 100 solution in PBS. Nile red fluorescence in the nanoparticles present in the cell lysate was measured spectrophotometrically. The data is shown as mean±S.D. for n=6. * indicates p<0.01 compared with PBS dispersion.

FIG. 19. Nanoparticle content (μg/mg of protein content) observed in cell lysate after incubation time of 5 minute, 60 minute, and 3 hours. HCET cells were plated in a 48 well plate (surface area 0.95 cm²). At 80% confluency, nile red loaded nanoparticles entrapped in hydrogel or dispersed in phosphate buffer saline (PBS) were added to the wells. After the incubation (5 minute, 60 minute, and 3 hours), cells were lysed using 2% Triton X 100 solution in PBS. Nile red fluorescence in the nanoparticles present in the cell lysate was measured spectrophotometrically. The data is shown as mean±S.D. for n=6. * indicates p<0.01 compared with PBS dispersion.

### DETAILED DESCRIPTION

The invention provides photoactivatable dendrimers and hydrogels formed from the dendrimers. The dendrimer hydrogel (DH) possesses many unique structural characteristics and desirable properties, For example, proper selection of components results in dendrimers that are highly branched nanoparticles with a number of surface groups and charges. As described herein, the dendrimer hydrogel network allows for simultaneous delivery of both hydrophobic and hydrophilic drugs as needed. In particular, in one embodiment, the interior hydrophobic core of the dendrimer can encapsulate hydrophobic compounds, thus increasing their water solubility and loading amounts, while the cross-linked polymer network can load hydrophilic drugs. Photoactivatable dendrimer solutions are light sensitive, and are able to become viscous solutions and/or form a dendrimer hydrogel (DH) in situ upon light exposure. DH exhibits pH-dependent degradation responsiveness, controllable release kinetics and swelling behavior. Importantly, DH has demonstrated good mucoadhesiveness, making possible sustained drug release, and has favorable biological properties, such as non-toxicity. Further, this new platform integrates the structural characteristics and properties of in situ gelling, mucoadhesive, and nanoparticle delivery systems, representing a new generation of hydrogels.

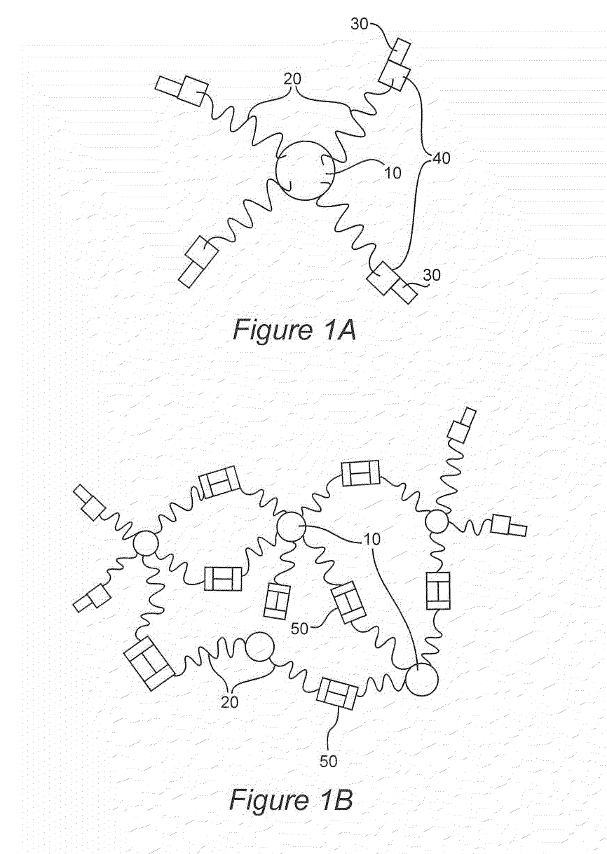

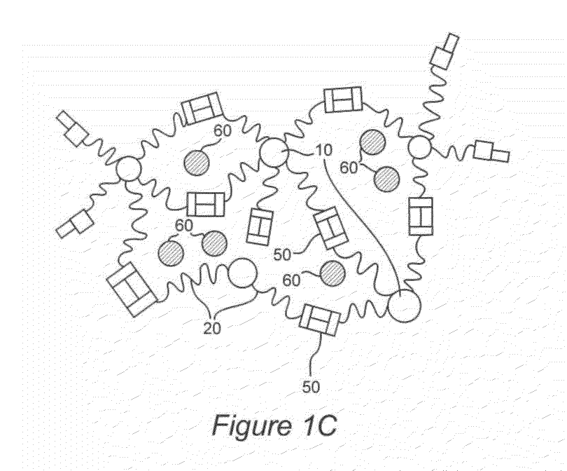

Individual photoactivatable dendrimers comprise a dendrimer to which a plurality of polymer chains have been conjugated. Reactive photoactivatable groups are attached to the terminal end of the polymer chains (i.e. the end that is not conjugated to the dendrimer). A generic photoactivatable dendrimer is depicted in FIG. 1A, where polymer chains 20 are shown as conjugated to dendrimer 10. Photoactivatable groups 30 are shown as attached to functional groups 40 located at terminal ends of the conjugated chains. It should be understood that during attachment of a photoacitvatable group 30 to a functional group 40, the functional groups may be modified, e.g. by loss of one or more atoms, in order to form a bond (usually covalent) with the photoacitvatable group. For example, if the “functional group” located at a terminal end of a polymer chain is a hydroxyl (OH), during attachment of a photoacitvatable group, H may be lost and a covalent bond to O may be formed. A crosslinked hydrogel of this type is depicted schematically in FIG. 1B, where polymer chains 20 conjugated to dendrimers 10 are shown with intervening photoactivated crosslinkages (crosslinked groups) 50.

In some embodiments of the invention, the hydrogels further comprise nanoparticles dispersed therein. This embodiment is illustrated in FIG. 1C, which shows the hydrogel of FIG. 1B with dispersed nanoparticles 60. This embodiment of the invention is discussed in detail below.

Examples of dendrimers that may be used in the practice of the invention include but are not limited to amine-terminated PAMAM dendrimers such as G3.0, carboxylate-terminated PAMAM dendrimers such as G3.5, hydroxyl-terminated PAMAM dendrimers, PAMAM dendrimers having a mixed amine/hydroxyl surface, poly(propyleneimine) (PPI) dendrimers, polylysine dendrimers, etc.

Polymer chains which may be attached to the dendrimer include but are not limited to PEG or polyethylene oxide (PEO), PEG or PEO-containing block copolymers including poly lactic acid (PLA)-PEG, PEO-PPO-PEO, polylysine, silicone, proteins, antibodies, growth factors, etc. Depending on the type of polymer chain that is used, the length of the chains may be the same or they may vary. Generally a polymer chain will be of a length in the range of from about 300 daltons (Da) to about 100000 Da, and will extend out from the dendrimer sufficiently to allow further modification of the terminal functional groups of the chains, and to allow sufficiently diverse crosslinking to faun a suitable hydrogel. Polymer chains may be of the same length or of differing lengths. If the polymer chains are PEG, the sizes of PEG that are used will generally be in the range of from about 1500 Da to about 20000 Da, depending on the number of PEG on the surface, dendrimer generation, concentration of PEGylated dendrimer in solution, etc. Particularly, a G3.0 PAMAM dendrimer fully conjugated with PEG 12000 generates a stable crosslinked dendrimer hydrogel network. In addition, in some embodiments of the invention, a mixture of different types of polymer chains may be conjugated to the dendrimer. By “conjugated” we mean that the polymer chains are chemically attached or bonded to the dendrimer, e.g., by covalent bonding. Those of skill in the art will recognize that the exact chemistry that is used to attach polymers to the dendrimers will vary from polymer to polymer, depending on the type of reactive groups that are present in the dendrimer and the conjugatable end of the polymer. For example, the dendrimers may contain reactive groups such as amine or carboxylate which can react with or be modified to react with polymer reactive groups such as nitrophenyl chloroformate or hydroxyl. Generally, a density (e.g. an average density or number) of polymer chains of more than about 50% terminal groups per dendrimer is sufficient to prepare the photoactivatible dendrimers of the invention.

The polymer chains used to prepare the photoactivatible dendrimers bear on their non-conjugated ends (referred to herein as the “terminal” end of the chain, i.e. the end that is not attached to the dendrimer), either a photoactivatable group, or a functional group that is capable of binding to a photoactivatable group. For example, PEG contains terminal hydroxyls to which photoactivatable groups may be attached. By “photoactivatable group” we mean a chemical functional group that, upon exposure to a suitable wavelength/energy of the electromagnetic spectrum, is converted to a reactive species capable of forming covalent bonds with other similarly reactive species of the same kind or a different kind. Suitable photoactivatable groups include but are not limited to acrylate, aryl azides, phenyl azide, fluorinated aryl azides, benzophenones, diazo compounds, diazirine derivatices, etc. In order to provide sufficient photoactivatable groups per dendrimer for crosslinking to other dendrimers (described below), typically at least about 25%, preferably about 50%, more preferably about 75%, and most preferably 90-100% of the polymer chains attached to a dendrimer will contain an attached photoactivatable group.

Those of skill in the art will recognize that the choice of photoactivatable groups will be predicated, in part, on the application of the photoactivatable dendrimers, and hydrogels formed therefrom. For example, if the hydrogel is cured in vitro, then any wavelength may be used since there need not be any concern about damaging living tissue. However, if the hydrogel is to be cured in or on a living being, care is taken to utilize photoactivatable groups which can be activated under conditions that are not that harmful or that are minimally harmful to living tissue. For example, crosslinked hydrogel triggered by acrylates has been found to be minimally toxic.

Upon exposure to a suitable wavelength of light, in the presence of a photoinitiator, the photoactivatable groups become crosslinked to one another, thereby covalently linking adjacent dendrimers to each other and forming a hydrogel. Exemplary photoinitiators for use in this step include but are not limited to dimethoxyphenyl acetophone, Irgacure 2959, eosin Y mixed with triethanolamine and 1-vinyl-2 pyrrolidinone, etc. If the step is carried out in living tissue, physiologically compatible photoinitiators such as eosin Y mixed with triethanolamine and 1-vinyl-2 pyrrolidinone are employed.

Those of skill in the art will recognize that both the wavelength of light and the necessary time of exposure of the dendrimers to the light will vary depending on several factors, e.g. how much hydrogel is being formed, the desired extent of crosslinking, the environment in which the reaction takes place (e.g. temperature, amount of water present, etc.), and other factors. In particular, the type of photoactivatable group may dictate the amount of light energy that is required (both wavelength and time of exposure). Preferably, especially if crosslinking (curing) of the dendrimers to form the hydrogel is carried out in or on living tissues, the time should be minimized, e.g. preferably to less than about 10 minutes, and more preferably to less than about 5 minutes, e.g. for 1, 2, 3, 4 or 5 minutes. In some embodiments, the curing hydrogel may be exposed to different types of radiation, e.g. to ultraviolet light and to natural sunlight, either simultaneously or sequentially. For other applications (e.g. to prepare delivery systems for medicinal purposes), the curing time and conditions may be much longer/harsher.

The extent of crosslinking, which determines the pore size of the hydrogel, can be varied or fine-tuned according to the intended application of the hydrogel. Pore size ultimately determines the ease with which substances can enter (diffuse into) the interior of the hydrogel and how deeply into the interior a substance can penetrate in a given amount of time. For example, the pore size or crosslink density can be varied by adjusting the ratio of the concentration of photoactivatible dendrimers to that of photoinitiator.

In some embodiments, for example, in order to modulate the rate of degradation of the hydrogel, one or more secondary polymer component may be added to enhance the stability of the network. For example, linear polymers such as PEG, polypeptides, and proteins can be incorporated to form polymeric semi-interpenetrating network (semi-IPN). Linear polymers of appropriate amounts are mixed with photoactivatible dendrimers and subject to light exposure to form semi-IPN hydrogel. The degradation rate of the semi-IPN hydrogel can be varied. It is affected by the degradability and loading density of the incorporated linear polymers. Faster degradation will be enabled if proteins such as gelatin are encased as the secondary polymer component.

By selecting suitable dendrimer-polymer chain combinations, it is possible to prepare multifunctional photoactivatable dendrimers, e.g. those with one or more functional groups for any of several purposes. For example, in addition to sequestering substances of interest within the hydrogel, such substances may also be chemically attached to functional groups, e.g. carboxylates or other groups that remain on the dendrimer surface after conjugation of the polymer chains. The ionic properties, pH responsiveness, etc. of the hydrogels can be varied by selecting suitable dendrimers and/or polymer chains with desired functional groups, e.g. charged groups that are reactive, and/or which become protonated/deprotonated at a desired pH, thereby changing the physicochemical properties of the hydrogel, its degradation rate, swelling behavior, drug release kinetics, etc.

Due to the many advantages of the dendrimers and hydrogels of the invention, they have a wide variety of useful applications. For example, in the field of medicine, the hydrogels may be used to deliver medicaments and other beneficial substances. Because the interior of the dendrimers is generally hydrophobic, while the polymer chain portion of the gel is generally hydrophilic, both hydrophobic and/or substances, or amphiphilic substances, can be loaded into a hydrogel. Upon contacting the hydrogel, the substances will migrate into the interior of the hydrogels and partition into the environment that is most compatible with respect to charge, hydrophobicity, hydrophilicity, etc. Upon placement of a loaded hydrogel at a suitable location, the substances contained therein can then be delivered from the hydrogel to a desired site of action. Because substances within the hydrogel must then exit the gel by migrating through the pores, the hydrogels provide an excellent means for the extended delivery of substances over time, e.g. for controlled release of an agent of interest. As such, the hydrogels may be formulated for use in any of a variety of delivery modes, e.g. as capsules, tablets, lozenges, in patches, gels for topical or other applications.

This new material can be used for drug delivery and controlled release. Dendrimer hydrogels having carboxylate surface groups can be used to formulate dosage forms for oral drug delivery. Dendrimer hydrogel having primary amine surface groups can be used for ocular drug delivery. Dendrimer hydrogels containing amine groups can be used for sustained gene delivery and release for tissue engineering applications. In addition, this material also demonstrates good tissue adhesiveness. Ocular or other wound dressings can be developed based, for example, on amine group-bearing hydrogels. For example, Cyclosporine A, used for dry eye syndrome, can be loaded and released over an extended period of time by this new hydrogel type. Further, the release kinetics can be controlled by pH adjustment.

Dispersion of Nanoparticles within the Hydrogel

In some embodiments of the invention, the hydrogels described herein further comprise nanoparticles dispersed within the hydrogel. Generally, the nanoparticles contain or include at least one agent of interest, e.g. a biologically active agent such as a drug or therapeutic. The nanoparticles that are used in the practice of the present invention generally exhibit dimensions in the range of from about 1 nm to about 1000 nm, for example, in the range of from about 10 to about 500 nm, or from about 100 to about 200 nm, in a longest dimension, e.g. a diameter. Those of skill in the art will recognize that the nanoparticles described herein will generally be substantially spherical (hence size may be expressed in terms of a “diameter”), although this need not always be the case, as individual nanoparticles may vary somewhat (e.g. to be somewhat ovoid, or flattened, etc.), without effecting the practice of the invention.

The preparation of nanoparticles from polymers is well known in the art, examples of which include but are not limited to: emulsification-diffusion, salting-out, solvent displacement, emulsion evaporation, single oil-in-water (O/W) emulsion/solvent evaporation, etc. Those hyperbranched and dendritic polymers at the nanoscale are also classified as nanoparticles. Methods of nanoparticle preparation are described, for example, in issued U.S. Pat. No. 7,648,959 (Bender et al.) the complete contents of which is hereby incorporated by reference; and also in other issued US patents including U.S. Pat. Nos. 7,879,819; 7,867,556; 7,767,249; 7,713,551; 7,674,816; 6,506,405; 6,537,579; and 5,916,596; the complete contents of each of which is hereby incorporated by reference. Herein, Example 11 provides a description of one particular method for fabricating nanoparticles and dispersing them within a hydrogel.

Nanoparticles can be added to the hydrogel prior to a step of crosslinking so that the individual dendrimers crosslink around the nanoparticles, sterically trapping them within the gel matrix. In other words, the nanoparticles are actually added to a reaction mixture containing cross-linkable dendrimers and the reaction mixture is then crosslinked. Alternatively, nanoparticles can also be added after hydrogel formation. In this embodiment, viscosity of the gel matrix is designed so as to be of a suitable viscosity to retain the nanoparticles within the hydrogel matrix. For example, PAMAM dendrimer G3.0 when coupled with 3-4 PEG (Mr about 12,000 Da) acrylate chains forms a suitably viscous solution at a concentration of 8.1% w/v in the presence of an eosin Y-based photoinitiator (5:100 v/v) upon UV light treatment for 30 minutes (see Example 10). In other embodiments, depending on the exact composition of the nanoparticles, the nanoparticles themselves may be crosslinked or chemically bonded (e.g. covalently bonded, or held by ionic or hydrophobic interactions) to one or more dendrimers or polymer chains and hence at least partially immobilized or localized within the gel matrix (e.g. if the bond is not covalent, some diffusion of the particles may occur) or fully immobilized within the gel matrix (e.g. if a covalent bond is formed). Hence, as used herein “dispersed” within the hydrogel may refer to a true dispersion (e.g. a colloid or colloid-like mixture) or may be synonymous with “located” or “distributed” or “suspended” within the hydrogel. Generally, the nanoparticles are distributed throughout the hydrogel more or less evenly, e.g. the concentration of nanoparticles within the hydrogel is generally constant or uniform throughout the hydrogel, similarly to the distribution of particles in a colloidal gel.

The nanoparticles used in the practice of the invention generally are comprised of biocompatible, biodegradable polymers. Thus, bioactive agents contained within the nanoparticles may leave the nanoparticles both by simple diffusion or leaching out of the nanoparticle and into the surrounding milieu (in this case, hydrogel, and from the hydrogel into a targeted site of action) and/or may be released into the surrounding area due to breakdown of the nanoparticles and/or the hydrogel. In either case, delivery of the agent(s) of interest to the surrounding area is generally slow, e.g. on the order of several hours, such as at least 1, 2, 3, 4, 5, 6, 7, 8, 9, 10, 11, 12, 13, 14, 15, 16, 17, 18, 19, 20, 21, 22, 23, or 24 hours, or even several days, such as 1, 2, 3, 4, 5, 6, or 7 days, or possibly one week or more. In fact, the rate of release can be purposefully modulated by selecting particular nanoparticle compositions, e.g. by selecting relatively stable and/or less porous polymers for longer duration, or less stable and/or more porous polymers for more rapid egress of the agent.

Exemplary biocompatible, biodegradable polymers are well known by persons skilled in the art, as are methods for selecting polymers with desired properties for a particular application (e.g. loading potential, delivery rate, etc.). Examples include but are not limited to: polyesters from hydroxycarboxylic acids such as poly(lactic acid) (PLA), poly(glycolic acid) (PGA), polycaprolactone (PCL), copolymers of lactic acid and glycolic acid (PLGA), copolymers of lactic acid and caprolactone, polyepsilon caprolactone, polyhyroxy butyric acid and poly(ortho)esters, polyurethanes, polyanhydrides, polyacetals, polydihydropyrans, polycyanoacrylates, natural polymers such as alginate and other polysaccharides including dextran and cellulose, collagen, albumin, chitosan, hyaluronic acid, etc. In some embodiments, the nanoparticles are designed to actually enter cells at the site where the hydrogel-nanoparticle dispersion is applied. In this embodiment, the composition of the nanoparticle is tailored so as to enhance certain desirable properties such as mucoadhesiveness, biodegradability, abundance of amine surface groups, excellent biocompatibility, etc. Further, the size of the nanoparticles may be crucial, with, for example, nanoparticles made with PLGA (Mr of 30,000-35,000 Da) being a suitable choice. However, in other embodiments, the PLGA may range from about 2,000 to about 100,000 Da, e.g. about 5,000; 10,000; 15,000; 20,000; 25,000; 30,000; 35,000; 40,000; 45,000; 50,000; 55,000; 60,000; 65,000; 70,000; 75,000; 80,000; 85,000; 90,000; or 95,000 Da.

Generally, in the practice of the invention, an agent of interest, e.g. a bioactive agent such as a drug or medicament, is “loaded” or incorporated into the nanoparticles prior to dispersion of the nanoparticles into the hydrogel. For those nucleic acid therapeutics such as DNA plasmid, siRNA, shRNA, etc, they can be either encapsulated into nanoparticles or complexed with nanoparticles such as amine-terminated dendrimers through electrostatic interactions. Those of skill in the art are aware of methods of incorporating such agents into nanoparticles (e.g. see the issued US patents referenced above), and are familiar with calculating suitable concentrations of agents, and of determining e.g. a suitable rate of release from the nanoparticles so as to accord with treatment goals, with location of delivery, etc.; and of determining the compatibility of the agent(s) of interest and the components which make up the nanoparticles (e.g. hydrophobicity, hydrophilicity, permeability, etc.).

In some embodiments, a single type of nanoparticle is dispersed within the hydrogel, but this is not always the case. The invention also encompasses hydrogel-nanoparticle dispersions in which a plurality of different types of nanoparticles are dispersed, e.g. nanoaparticles with differing compositions, or which contain different bioactive agents, or which have different release of absorption properties, or differing rates of biodegradation, etc.

Bioactive agents which may be incorporated into the hydrogel-nanoparticle dispersions of the invention include but are not limited to small-molecular-weight drugs, protein and polypeptide therapeutics, nucleic acid therapeutics such as DNA plasmid, siRNA or shRNA, metal-based drugs, dyes or fluorescent molecules for treatment, diagnosis, or imaging, etc.

Because of the relative immobility of the hydrogel after administration (in other words, the hydrogel tends to stay at the site where it is applied; it is not a free-flowing liquid), the hydrogels of the invention are well suited to the delivery of drugs to a particular site of interest where a long-acting effect is desired. Exemplary targeted sites include but are not limited to, for example, wounds, burns, etc. at a surface to which the hydrogel can be applied. The hydrogel-nanoparticle dispersions of the invention are especially well-adapted for administration to the eye, and hence are especially useful for the treatment of eye conditions or diseases such as glaucoma, dry eye syndrome, eye infections, eye irritations (e.g. caused by contact lenses, exposure to chlorine, smog or other irritants, etc. and provide sustained release and enhanced bioavailability to the eye, and dramatically improve patient compliance, particularly among patients suffering from chronic ocular diseases, etc.

In one embodiment, the disease that is treated is the eye disease glaucoma, and active anti-glaucoma agents such as one or both of brimonidine and timolol (or suitable salts thereof such as timolol maleate) are delivered. In this embodiment, the quantity of e.g. timolol maleate loaded into the nanoparticles is generally in the range of from about 2.5% to 5%, or in the range of from about 3.0% to about 4.0% (e.g. about 3.1, 3.2, 3.3, 3.4, 3.5, 3.6, 3.7, 3.8, or 3.9%), and preferably 3.5% for timolol maleate. For brimonidine, the range is typically lower, e.g. from about 0.1% to about 1.5%, or from about 0.5% to about 1% (e.g. about 0.6, 0.7, 0.8, 0.9%), and preferably 0.75%. Further, the amount of drug per volume of the nanoparticle material is, when PLGA nanoparitcles are being loaded, generally in the range of from about 20 to 60 mg, or about 30 to 50 mg (e.g. 30, 35, 40, or 45 mg) and preferably 40 mg of timolol maleate per 100 mg of PLGA; and in the range of from about 5 to 40 mg, or from about 10 to about 30 mg (e.g. about 15, 20, or 25 mg), and preferably is about 20 mg per 100 mg of PLGA. For such combinations, the PLGA is typically dissolved in a solvent such as dichloromethane (DCM, 100 mg/ml) and the drugs are mixed into the PLGA-DCM solution, which is then further mixed with 2% polyvinyl alcohol (1:10 v/v).

Nanoparticles are dispersed in the hydrogel as described above. Various suitable ratios of nanoparticles to hydrogel may be employed, depending, for example, on the use of the preparation. For example, in embodiments for which delivery to the eye is contemplated, a mass ratio of e.g. PLGA to dendrimer hydrogel of about 0.5:25, or about 0.75:20, and preferably about 1:16.2 may be suitable for dispensing via a dropper. Other ranges (e.g. from 0.1-99% of either hydrogel to nanoparticle or nanoparticle to hydrogel may be suitable.

In some embodiments, the hydrogel-nanoparticle dispersion composition may be delivered directly to a targeted area without further preparation, e.g. when the targeted area is a surface wound, a “pocket” in the gum, the vagina, or some other relatively readily accessible area. In other embodiments, the composition may be suspended in a physiologically compatible buffer (e.g. normal saline) in order to facilitate dispensing, e.g. with a dropper into the eye, into the ear, etc.

The hydrogel-nanoparticle dispersions of the invention are especially well-suited for use in the treatment of glaucoma since the dispersions provide extended release of drugs that are used to treat the disease. There is currently no cure for glaucoma. Thus, treatment is always long-term for the entire life of the patient. Current treatment regimens usually involve the administration of anti-glaucoma agents directly to the eye, e.g. with eye drops, which must be administered frequently, e.g. 3-4 times per day, and even more frequent administration may be optimal since the drugs are released immediately into the eye, and the effect last only a few hours at best. For many patients, this treatment regime is extremely inconvenient and doses of drug are likely to be missed. Many cases of glaucoma occur in the elderly, who are especially likely to forget to use the drops. Hence, poor compliance with medications is a major reason for vision loss in glaucoma patients. There is therefore a great need to develop drug formulations that provide sustained or prolonged delivery and release of anti-glaucoma agents directly to the eye.

Example 11 below shows data that was obtained using the hydrogel-nanoparticle dispersion of the invention for the in vivo delivery of two front line antiglaucoma drugs to the eye in a sustained manner. The data showed that application of a hydrogel-nanoparticle dispersion in which the nanoparticles comprised timolol and brimonidine directly to the eye resulted in the slow release of the drugs to the eye at clinically relevant levels for up to about one week. Obviously, a dosing regimen limited to only once per day or once every few days, and particularly if limited to once per week (or even longer time periods), would be much more convenient and easy for patients or caregivers to remember, would result in much higher levels of compliance, and hence improved clinical outcomes. In addition, the benefits of this slow release composition are not limited to the treatment of glaucoma, but may be extended to the treatment of any eye condition or disease, or to the treatment of any condition or disease which requires or could benefit from the sustained release of active agents.

Exemplary anti-glaucoma agents that may be delivered using the compositions and methods of the present invention include but are not limited to prostaglandin analogs such as latanoprost (Xalatan), bimatoprost (Lumigan) and travoprost (Travatan); topical beta-adrenergic receptor antagonists such as timolol, levobunolol (Betagan), and betaxolol; alpha-2-adrenergic agonists such as brimonidine (Alphagan); sympathomimetics such as epinephrine; miotic agents (parasympathomimetics) like pilocarpine; carbonic anhydrase inhibitors like dorzolamide (Trusopt), brinzolamide (Azopt), and acetazolamide; physostigmine, etc.

In another embodiment, the invention provides a method for forming a dendrimer hydrogel. The method includes several steps, the first of which is the covalent attachment of photoactivatable reactive groups terminal diol moieties of a plurality of polyethylene glycol (PEG)-diol polymer chains. Exemplary photoactivatable reactive groups include acrylate. This reaction transfers photoactivatable reactive groups to one or both termini of PEG chains and produces photoactivatable PEG polymer chains which are photoactivatable by virtue of the presence of at least one photoactivatable group located at one terminus or both termini of each chain. The second step is the attachment of the photoactivatable PEG polymer chains to a plurality of dendrimers (for example, PAMAM dendrimers such as PAMAM G3.0). The attachment to a dendrimer can occur only if the PEG polymer has a free terminus, i.e. a terminus that was not modified by attachment of a photoactivatable reactive group. The free hydroxyl itself or through a chemically activated form reacts with a chemically reactive group on the dendrimer, for example, amine or carboxylate, etc. Typically, an average of about 1-10, e.g. about 2, 3, 4, 5, 6, 7, 8, 9, or 10 polymer chains are attached per dendrimer, and preferably from about 3 to about 4 polymers are attached to each dendrimer. Once sufficient polymers are attached per dendrimer, unreacted polymers are removed, and the mixture of dendrimers with attached photoactivatable PEG polymer chains is exposed to a wavelength of light suitable to cause or initiate cross-linking between terminal photoactivatable reactive groups of the PEG polymer chains, thereby linking the PEG polymer chains to each other. The linking of polymer chains produces a network of interlinked dendrimers (i.e. a dendrimer hydrogel) which are connected to each other via the polymer chains. By first attaching the photoactivatable reactive groups to the polymer chains, attachment of the photoactivatable reactive groups to the dendrimers themselves is prevented. Thus, during the photoinitiated crosslinking step, polymer chains link only to one another, and not back to the dendrimer. This method thus provides exceptional control over the extent of crosslinking of the hydrogel, and hence control over the properties of the hydrogel (viscosity, porosity, degradation, etc.), and keeps the dendrimer surface available for conjugation of drug molecules or any other molecules of interest.

The invention also provides methods for treating individuals or subjects with conditions or diseases that can be ameliorated by the application of a dendrimer hydrogel or a dendrimer hydrogel/nanoparticle dispersion, especially when the DH or the nanoparticles delivery a bioactive agent of interest to a desired location to treat the condition or disease. Such methods may involve identifying individuals with symptoms of a condition or disease that can be treated in this manner, administering a DH or DH/nanoparticle dispersion to a suitable location in or on the subject, and allowing the delivery material to remain at the site long enough to deliver a bioactive agent of interest from the DH or DH/nanoparticle dispersion to the site. The site that is treated may be any that is or that can be made accessible to the DH or DH/nanoparticle dispersion, e.g. the eye, skin, wounds, ears, vagina, surgical incisions, etc. While subjects who are treated in this manner are frequently mammals (e.g. humans), this is not always the case. For example, veterinary applications of this technology (e.g. treatment of companion animals, livestock, and animals in captivity, etc.) are also encompassed by the invention, and treatment protocols may extend to non-mammalian species as well. The sustained delivery of agents by the methods of the invention are, in fact, highly suitable for the treatment of animals since fewer applications are necessary to achieve a desired effect. Further, the DHs or DH/nanoparticle dispersions of the invention may also be used for research purposes.

Various exemplary embodiments of the invention are further illustrated in the ensuing examples, which should not be considered limiting in any way.

### EXAMPLES

### Example 1

### Preparation of Photoactivatable Dendrimers

### Introduction

Hydrogels are crosslinked insoluble network of polymer chains that swell in aqueous solutions, which have found many applications including drug delivery and tissue regeneration. Dendrimers provide an ideal platform for drug delivery as they possess a well-defined highly branched nanoscale architecture with many reactive surface groups. Their highly clustered surface groups allow for targeted drug delivery and high drug payload to enhance therapeutic effectiveness. This example describes a new type of polyionic hydrogels based on dendrimers with applications in drug delivery and tissue engineering. Polyethylene glycol (PEG) was first conjugated to the Starburst™ G3.0 PAMAM dendrimer to form stealth dendrimers through one ending site of PEG using p-nitrophenyl chloroformate (4-NPC) and triethylamine (TEA). The free hydroxyl group of PEG was further converted to an acrylate group using acrolyl chloride and triethylamine. The conjugation was characterized with ¹H-NMR. The ninhydrin assay was used to estimate the loading degree of PEG on the dendrimer surface. The molecular weight and loading degree of PEG was varied. Hydrogel formation was realized by subjecting dendrimer-PEG acrylate to UV exposure for a brief period of time in the presence of dimethoxy-2-phenyl-acetophenone (DMPA) photoinitiator. Viscosity increase was observed after hydrogel formation. PEGylated G3.0 PAMAM dendrimer served as cross-linking agent to form hydrogels because of its multiple functionalities. The surface charges conferred by terminal groups on the dendrimer surface made the hydrogel polyionic with controllable charge density. This new type of hydrogel has many favorable biological properties such as non toxicity and non immunogenecity and multifunctionalities for a variety of in vivo applications. The current studies have demonstrated the feasibility of chemistry and hydrogel formation, and uses include drug delivery via drug encapsulation in a hydrophobic dendrimer core, and later release in a controlled fashion.

### Conjugation of PEG to Full Generation PAMAM Dendrimer G3.0

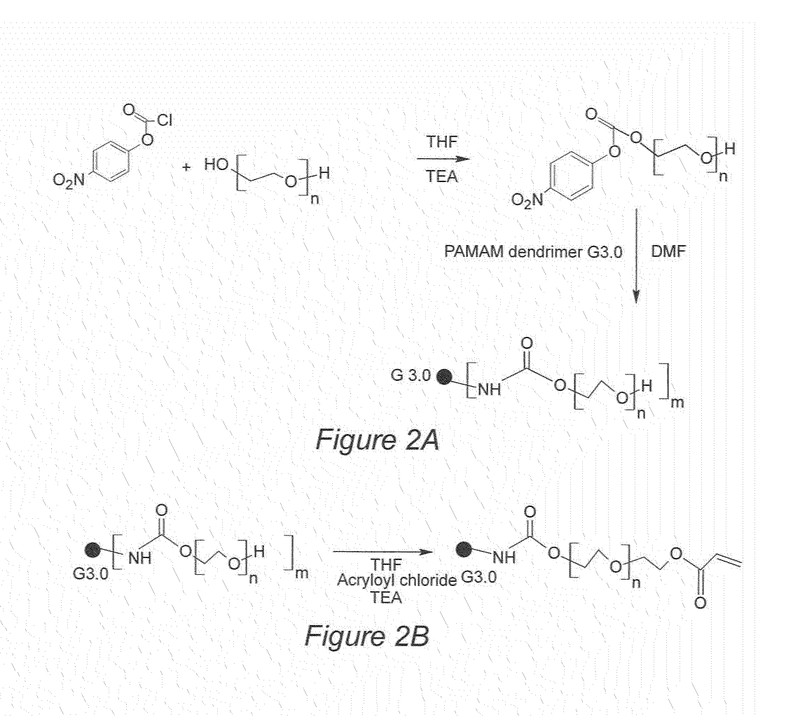

As illustrated in FIG. 2A, one hydroxyl end group of PEG diol (3 different molecular weights used 1500, 6000 and 12000 Da) was activated first with 4-NPC and TEA to form OH-PEG-NPC conjugates. Briefly 0.4 mmol of PEG was dissolved in 40 ml of THF. To this solution 0.45 mmol (80.6 mg) of 4-NPC and 0.4 mmol of TEA were added dropwise. The mixture was stirred for 24 hrs, and then centrifuged at 10 rpm for 10 minutes to filter off the salt. The supernatant was precipitated in ethyl ether (40 ml) and kept at −20° C. for further precipitation. After 24 hrs, the precipitate was collected and dried using freeze dry system (FTS) to obtain OH-PEG-NPC conjugates. OH-PEG-NPC was then reacted with PAMAM dendrimer generation 3.0 (where the molar ratio of PEG-NPC/dendrimer was 32:1) in dimethylformamide (DMF) for 72 hours forming PEGylated dendrimer conjugate. This solution was precipitated in 50 ml of ethyl ether and kept at −20° C. for further precipitation. The precipitate was collected and freeze dried with FTS 58. Dialysis was carried out to remove excess of PEG for further purification of the product. The resulting G3.0-PEG-OH was then freeze dried. The degree of PEGylation on the dendrimer as well as the molecular weight of G3.0-PEG-OH was characterized with ninhydrin assay and ¹H-NMR spectroscopy.

### Conjugation of PEG to Half Generation PAMAM Dendrimer G3.5

Conjugation of PEG to half generation PAMAM G3.5 involved the activation of carboxyl (—COOH) groups of the dendrimer using dicyclohexylcarbodiimide (DCC) and 4-dimethylaminopyridine (DMAP). Prior to the reaction, 1 μmol of PAMAM G3.5 was dried via rotary evaporation. The obtained dry product was then dissolved in 1 ml of DI water. The solution was then acidified with 3 drops of 1 Normal HCl. The acidified solution was dried again by rotary evaporation and then re-dissolved in 2 ml of DMF. To this solution PEG diol was added, followed by the addition of DCC and DMAP where the feeding molar ratio of PEG diol:DCC:DMAP:G3.5 was 64:64:64:1. The solution was stirred for 24 hrs at 4° C. After 24 hours the solution was added dropwise to cold ether and kept at −20° C. for 24 hrs. The precipitate was obtained by centrifugation. Dialysis was carried out to remove un-reacted DCC, PEG, and DMAP for further purification of the product. The product thus recovered was G3.5-PEG-OH. The degree of PEGylation was then determined by ¹H-NMR.

Herein, PAMAM/PEG-based dendrimers are described using notations such as “X [Y—Z]ₙ” where X=the dendrimer type, Y=the polymer chain type, Z=the photoactivatable group, and n=the average number of polymer chains per dendrimer. For example, “G3.5-[PEG 1500-acrylate]43” represents a photoactivatable dendrimer where “G3.5” represents PAMAM dendrimer type (generation) “03.5”, “PEG 1500” represents PEG polymer chains with an average molecular weight of 1500 Da, “acrylate” is the photoactivatable group acrylate, and “43” indicates that the average number of conjugated PEG chains per dendrimer is 43.

### Conversion of Free Hydroxyl Group of PEG to an Acrylate Group

As shown in FIG. 2B, PEG diol was acrylated in order to make photo-initiated crosslinking reaction possible. To convert the free hydroxyl group of PEG on the dendrimer surface to an acrylate group, the reaction procedure involved the following reagents: dendrimer-PEG-OH, acrolyl chloride, and TEA at the respective molar ratio of 1:4:6. G3.0-PEG-OH was dissolved in 5 ml of tetrahydrofuran (THF). To this solution a mixture solution of acrolyl chloride and TEA was added dropwise and stirred for 4 hours. Then centrifugation was carried out to remove the salt and the supernatant was collected. The collected supernatant was added dropwise to 40 ml of ethyl ether and kept at −20° C. for further precipitation. The precipitate was extracted and dialyzed to make sure that excess of acrolyl chloride was removed. The resulting G3.0-PEG-acrylate was then freeze dried. G3.5-PEG-OH was converted to G3.5-PEG-acrylate following the same procedure as described above.

### Example 2

### Activation of Hydrogels: Sol-Gel Phase Transition Studies

In order to minimize the exposure of UV radiation for hydrogel formation, a combination of regular day light and UV radiation was studied. This study was carried out to determine the conversion from sol to gel phase over a period of time for dendrimer based hydrogel. G3.0-[PEG 12000]28 was used in this study. For this 7.5 wt % polymer was dissolved in 100 μL of distilled water. To this solution, 5 μL of photoinitiator Eosin Y system was added. 12 sample solutions were prepared. Then these solutions were allowed to cure under day light for 24 hrs, 48 hrs, 72 hrs and 1 week; three samples for each time period of curing. After 24 hrs, three samples were subjected to UV light for one minute, 5 minutes, and 10 minutes, respectively. The vials were inverted to determine the flow or no flow condition and the time after which the flow was seen. Similarly the samples exposed to regular day light for 48, 72 hrs, and 1 week were subjected to 1, 5, and 10 minutes of UV exposure and tube inversion was done to determine the sol-gel transition phase.

It was observed that 30 minutes of UV exposure was needed for hydrogel formation. In an attempt to cut down UV exposure, solutions of the polymer and photoinitiator were cured under regular day light for different time periods first, followed by UV exposure. Table 1 shows the results of Sol-gel phase transition studies. It was observed that UV exposure can be reduced to about 10 minutes with the utilization of combination of two sources of curing, regular day light and UV radiation. When the mixture of polymer and photoinitiator was allowed to cure in regular day light for a longer time period hydrogel formation can be realized with the UV exposure reduced to between 1 and 10 minutes. Linear PEG-acrylate was used as control, hydrogel formation was observed after 24 hours of curing in regular day light without any UV exposure.

TABLE 1
Sol-gel phase transition of G3.0-[PEG 12000-acrylate]28

| | Time cured in day-light | | | | | | | | | | | |
|---|---|---|---|---|---|---|---|---|---|---|---|---|
| | 24 hours | | | 48 hours | | | 72 hrs | | | 1 week | | |
| Time of UV exposure | 1 Min | 5 min | 10 min | 1 min | 5 min | 10 min | 1 min | 5 min | 10 min | 1 min | 5 min | 10 Min |
| Flow results after | −− | −− | − | (+) | (+) | + | + | + | ++ | ++ | ++ | ++ |
| tube inversion | | | | | | | | | | | | |

Instant Flow = −−, Flow after 5 seconds = −, Flow after 10 seconds = (+), Flow after 20 second = +, Flow after 30 seconds = ++

This example shows that the hydrogels of the invention can be cured in time frames that are compatible with physiological uses of the hydrogels.

### Example 3

### Swelling Tests

The prepared hydrogel were subjected to swelling test to evaluate the equilibrium water content (EWC) within the network. Water swelling experiments were conducted at room temperature at different pHs (i.e., 4.4, 2, 7.4, and 10). Prior to evaluation of equilibrium water content or calculation of the swelling ratio, the hydrogels were dried. Hydrogel samples were accurately weighed prior to immersion into the swelling media. The hydrogel samples were taken out periodically from the swelling media, blotted dry with an absorbent tissue and weighed. Each water swelling test was carried out over a period of 24 hours.

The second method utilized centrifuge tubes with a membrane having a molecular weight cut off of 300 Da. Each hydrogel sample was placed in the upper chamber of the tube having membrane and incubated in the medium. These centrifuge tubes containing hydrogel and the medium were centrifuged at predetermined time points. The medium was collected at the bottom of the tube. The hydrogel was weighed and swelling ratio calculated. The hydrogels were put back in the tubes after weighing and same procedure was carried out for 24 hours of incubation.

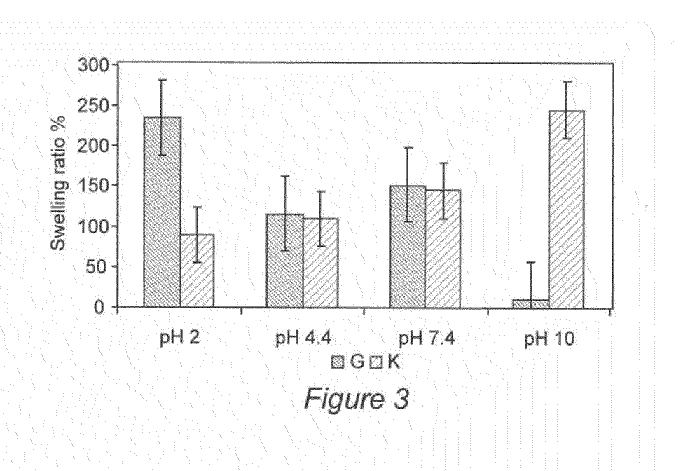

FIG. 3 gives a comparison of water swelling behavior between full generation (G3.0) and half generation dendrimer (G3.5) based hydrogel. The half generation dendrimer has carboxyl group on the surface. It was observed that at low pH (i.e. pH 2), half generation dendrimer based hydrogel showed a lower swelling ratio (89.9%) indicating less water absorption. It is assumed that the hydrogel network based on G3.5-[PEG 1500-acrylate]43 becomes hydrophobic at pH 2, and less water is absorbed while, at pH 10 the network becomes hydrophilic and absorbs more water (i.e., the swelling ratio of 246% at pH 10). Thus % increase in swelling ratio for G3.5-[PEG 1500-acrylate]43 from pH 2 to pH 10 is 95.73%. G3.0-[PEG 12000-acrylate]3+linear PEG 1500-acrylate shows high swelling ratio (234.3%) at pH 2 and lower swelling ratio (10%) at basic pH (i.e. pH 10). Thus % decrease in swelling ratio for G3.0-[PEG 12000-acrylate]3+linear PEG 1500-acrylate from pH 2 to pH 10 is 173.64%. However when G3.5-[PEG 1500-acrylate]43 hydrogels were compared with low PEGylated dendrimer G3.0-[PEG 12000-acrylate]3, the difference between swelling ratio at high and low pH was less pronounced because G3.5-based hydrogels prepared had 43 out of 64 carboxyl groups of G3.5 dendrimer that were conjugated with PEG as compared to 3 out of 32 amine groups conjugated with PEG for low PEGylated G3.0 dendrimer. The higher degree of PEGylation reduces the number of exposed surface groups that are responsible for pH sensitivity of the network.

This example illustrates that the hydrogels of the invention can be tailored to display pH sensitivity with respect to the degree of swelling. The hydrogels formed from full generation dendrimer can be implemented, for example, for ocular drug delivery. Since these hydrogels present with cationic charges, they would likely have longer retention on the anionic cornea through ionic interactions. This would help increase compliance and promote efficient drug delivery. Half generation dendrimer-based hydrogels can be used, for example, for oral drug delivery as they can react to pH gradient. Half generation dendrimer-based hydrogels have maximum swelling ratio at basic pH and hence a drug loaded into the hydrogel can be diffused out while at acidic pH, for example, within the digestive system.

### Example 4

### Cytotoxicity Testing

The cytotoxicity of the fluoroscein isothiocyanate (FITC)-conjugated G3.0-PEG was evaluated in vitro using cell line RAW264 mouse macrophages. RAW264 mouse macrophages (1×10³ cells/well) were seeded in a 24-well cell culture plate at 37° C. in 1 ml of medium (Dulbecco's Modified Eagle's Medium, DMEM) supplemented with 10% fetal calf serum, 100 UI/ml penicillin-streptomycin) in an atmosphere of 10% CO₂. After 24 h, the culture medium was replaced and different amounts of FITC-G3.0-[PEG12000-acrylate] 28 (Mw=34685) and cross-linked FITC-G3.0-[PEG12000-acrylate]28 (Mw=34685) were added. Their final concentrations were 0.2, 2, 20, 50, or 100 μM. The culture plate was then incubated at 37° C. in a tissue culture incubator for 2 days. After incubation at 37° C. for 2 days the medium was aspirated and 200 μL of trypsin solution was added to each well to prepare cell suspension solution. Then the cell suspension solution together with former medium was centrifuged at 3000 rpm for 3 min and the supernatant was discarded. The cells were re-suspended in 0.1 ml of phosphate buffered saline (PBS) or serum-free complete medium and to it 0.1 ml of 0.4% trypan blue solution added. The mixture was allowed to incubate 3 min at room temperature. Then a drop of trypan blue/cell mixture was placed onto a hemacytometer. The hemacytometer was then used to count cells. The unstained (viable) cells were then counted.

Cytotoxicity of the synthesized nanoparticle and nanomatrices were analyzed using RAW264 mouse macrophages cell lines. Uncrosslinked and crosslinked dendrimer-PEG displayed dose-dependent cytotoxicity; however, they had a negligible toxic effect on the cells at concentrations of 0.2 μM or below during an exposure period of 48 hours, showing 100% cell viability.

This example shows that the dendrimers and hydrogels of the invention are physiologically compatible with cells at concentrations and time periods that are clinically relevant.

### Example 5

### Adhesive Properties

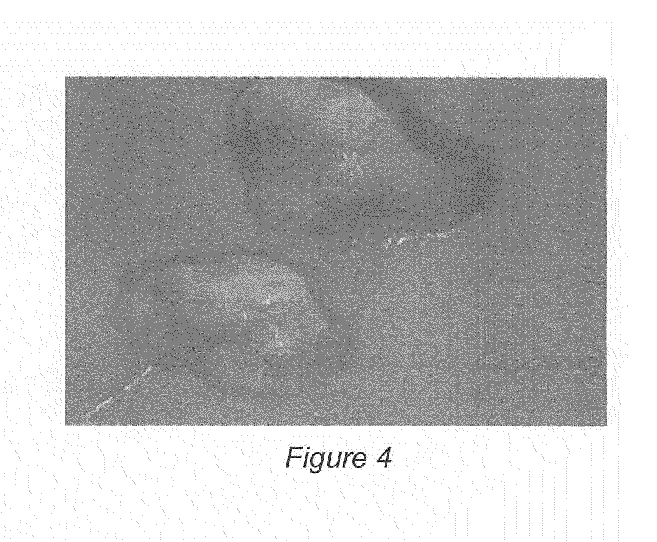

Dendrimer hydrogels were prepared and assessed for their adhesive abilities. The results showed that hydrogel preparations (e.g. G3.0-PEG 12000) bond well to a variety of substrates, in particular those of very low surface energy such as polytetrafluoroethylene (PTFE, see FIG. 4). In addition, the hydrogels exhibit a superior ability to hold (retain) water. For example, they keep their hydrated state for several months at ambient temperature in a fume hood. In addition, they retain an appreciable amount of water even after a long period of lyophilization under vacuum.

### Example 6

### Analysis of Drug Release Kinetics

To understand the mechanism of release of an active agent from the prepared hydrogels, drug Cyclosporine A, which is sparingly soluble in water, was used. Drug loading was based on water for forming drug incorporated hydrogel as follows. First the polymer (half generation dendrimer (G3.5-[PEG 1500-acrylate]) was dissolved in 100 μL DI water. To this solution excess mount of cyclosporine A was added. This solution was vortexed vigorously and incubated for 24 hours. After 24 hours the solution was centrifuged to remove the solids and the supernatant (saturated with cyclosporine A) collected and mixed with photoinitiator solution, then exposed to UV radiation. It was assumed that the drug would be incorporated within the core of dendrimer. The hydrogel was placed in a dialysis bag, and then immersed in 100 ml medium at different pHs (i.e., 2, 7.4, and 10) for 24 hours covered with parafilm and stirring constantly. Samples were taken from this solution at predetermined time intervals and analyzed using UV-V is spectrophotometer. The absorbance measured with UV-Vis spectrophotometer was compared with the standard curve of cyclosporine A and the concentration of the drug was determined. The total amount of drug released from the hydrogel sample was compared with the calculated amount of incorporated drug by measuring the absorbance of the solution of polymer and cyclosporine A prior to hydrogel formation.

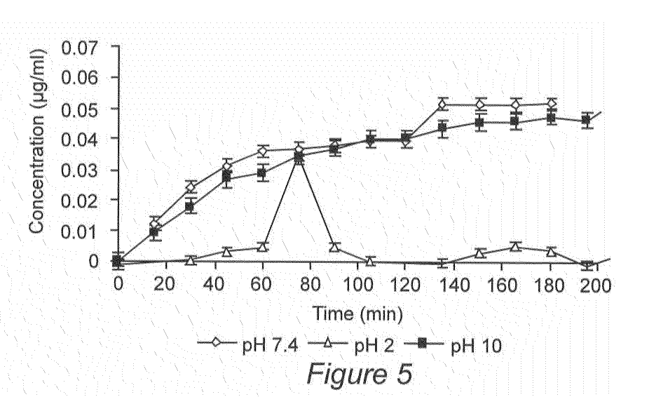

The release results are shown in FIG. 5. It was observed that the drug release had a high rate at pH 7.4 and pH 10 and a lower rate at pH 2. As both pH 7.4 and pH 10 were well above the pKa of carboxylate group on the dendrimer surface, the hydrogel had similar hydrophilicity at pH 7.4 and pH 10, resulting similar release rates for pH 7.4 and pH 10. Because the hydrogel became hydrophobic at pH 2, the release of drug was slowed down due to network shrinking.

This example shows that the rate of release of a drug loaded into a hydrogel of the invention varies in response to changes in pH.

### Example 7

### Ocular Delivery of Hydrogel to Treat Glaucoma

Rapidly increasing clinical need for treating eye diseases and the shortcomings of conventional dosage forms necessitate development of new and innovative ocular drug delivery approaches in order to increase the ocular bioavailability of topically applied drugs. To this end, new dosage founts, such as mucoadhesive gels, microparticles, and nanoparticles, are being extensively studied. With the significant increase in the number of ophthalmic drug prescriptions worldwide as predicted, finding ways to get therapeutic drugs to the eye effectively, safely, and conveniently is becoming more important than ever. New dosage forms should also provide sustained drug release and less invasive modalities to reduce frequent dosing and increase patient compliance, which are particularly beneficial to patients suffering from chronic eye diseases such as glaucoma.

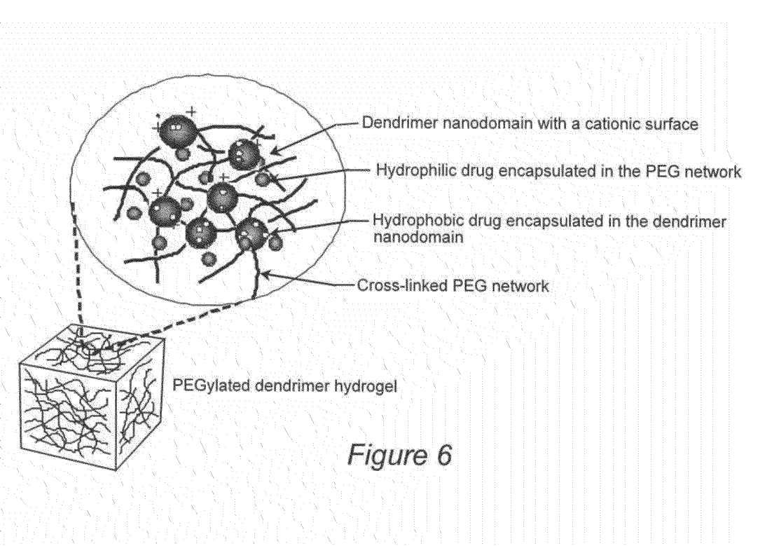

As described in Example 1, a novel highly adaptable and multifunctional polyamidoamine (PAMAM) dendrimer hydrogel platform with potential for ocular drug delivery has been developed. As illustrated in FIG. 6, in one embodiment the dendrimer hydrogel network consists of PAMAM dendrimer nanoparticles crossed linked with polyethylene glycol (PEG). New dosage formulations based on this dendrimer hydrogel enhance the bioavailability and/or prolong the therapeutic efficacy of antiglaucoma drugs such as brimonidine and timolol, hence reducing the dosing frequency to improve long-term patient compliance. Enhancing drug bioavailability and prolonging therapeutic efficacy is based on good mucoadhesiveness of the hydrogel, as well as its large loading capacity and sustained release capability.

Dendrimer hydrogel dosage forms for delivery of brimonidine and timolol are formulated. To ensure sufficient production consistency from batch to batch, several batches of dendrimer macromonomers at the gram-scale are prepared and characterized with routine analytical methods including ¹H-NMR (nuclear magnetic resonance), Fourier transform infrared (FT-IR), and gas phase chromatography (GPC). The formulations are shown to have the necessary properties to meet requirements for clinical use. In particular, physical, chemical, and microbiological parameters of the dosage formulations are considered, analyzed, and/or adjusted, including pH, osmolarity, mucoadhesiveness, drug release kinetics, degradation, toxicity, and sterility.

Sustained delivery and efficacy of the two antiglaucoma drugs is demonstrated with the aid of the dendrimer hydrogel dosage form. Dendrimer hydrogel (DH) solutions containing 0.1, 1, or 5% brimonidine (referred to as DH brimonidine) and dendrimer hydrogel solutions containing 0.25, 1, or 5% timolol (referred to as DH timolol) (n=6) are prepared. Dendrimer hydrogel solutions are made to have 1, 2.5, 7.5 wt % dendrimer macromonomers in deionized water, which can undergo gel formation upon long-wavelength light exposure (e.g. 510 nm). Hydrochloric acid or sodium hydroxide is added to adjust pH as appropriate. Benzalkonium chloride 0.01% is added as preservative. An osmolarity of 250-350 mOsm and a pH of 7.4-8.0 (0.1%) found in Alphagan® P (0.1%) are also expected for brimonidine-containing dendrimer hydrogels. The pH of the timolol-containing dendrimer hydrogel solutions is approximately 7.0, and the osmolarity is 274-328 mOsm as found in Timoptic®. Finally the formulations are sterilized by autoclaving at 121° C., 15 psi for 20 minutes. Non-toxicity of the formulated dosages to human corneal keratocytes is confirmed by using the MTT (3-(4,5-Dimethylthiazol-2-yl)-2,5-diphenyltetrazolium bromide) assay. Prior to use, dendrimer hydrogel solutions are kept in the dark to prevent light-induced viscosity increase or cross-linking.

Rabbits are treated with the formulated dosages. DH formulations of timolol and brimonidine or their plain solution form are administered as a 30 microliter drop to one eye of New Zealand white male rabbits and animals are sacrificed at different time points. The eye tissues including cornea, aqueous humor, and iris-ciliary body are isolated and extracted for the drug using liquid-liquid extraction and the drug levels are quantified. Sustained drug delivery to the target tissues including iris-cililary body and aqueous humor are evaluated. For efficacy assessment, intraocular pressure (IOP) over time is measured. Drug levels persist up to a week in DH formulation groups but not in conventional control drug groups.

Further details of this type of experiment and the results obtained therewith are provided in Examples 10-17 below.

### Example 8

### Oral Drug Delivery

Because of their pH sensitivity, dendrimer hydrogels composed of carboxylate-terminated dendrimer cores are utilized for delivery of a variety of drugs through an oral administration route with improved patient compliance and treatment efficacy. Drugs that are delivered include but are not limited to proteins, genes, growth factors, small-molecular-weight drugs, peptides. Such formulations tend to shrink at low pH in stomach to prevent drug release and degradation and release of therapeutics occurs in the small intestine or colon as pH increases. Bioavailability of the delivered therapeutics is increased with this means.

### Example 9

### Gene Delivery and Targeted Drug Delivery

Recent progress has demonstrated the use of genes in treatment of genetic diseases, viral diseases, and cancer. The application of therapeutic genes is made possible only with the aid of vectors as genes themselves are unable to cross the cell membrane mainly because of their negative charge. A variety of vectors have been developed to aid the entry of therapeutic genes into somatic cells. Gene transfer vectors are divided into two categories: viral vectors and nonviral vectors. Viral vectors have evolved functions to move genes into cells efficiently, but safety concerns have restricted their practical application. Nonviral vectors have attracted considerable attention for gene transfer as they can potentially avoid toxicity and immunogenicity, provide high gene carrying capacity, achieve prolonged gene expression, and allow low-cost manufacturing. The main obstacle for delivery of genes by nonviral vectors is to obtain specificity for a target cell, tissue, or organ type. In addition, the low bioavailability of genes caused by nuclease digestion and short blood half-life also are problems that need to be overcome. The lack of adequate functions to overcome the post-endocytosis barriers is one of the major reasons making current nonviral vectors far less efficient than viral vectors.

“Proton-sponge” polymers, such as polyamidoamine (PAMAM) dendrimers, have been used to facilitate endosomal escape of polyplexes as they contain a large number of secondary and tertiary amines with a pKa at or below physiological pH. Those secondary and tertiary amines adsorb the protons released from ATPase and subsequently cause osmotic swelling and rupture of the endosome membrane to release the entrapped polyplexes. The dendrimer hydrogel, particularly composed of amine-terminated dendrimer cores, is able to entrap genes and allow for sustained gene release and improved transfection. In addition, the highly adaptable structure of the dendrimer hydrogel allows for construction of a targeted delivery system by covalently conjugating targeting ligands such as epidermal growth factor (EGF), cetuximab, folic acid, etc. to dendrimers while drugs are either encapsulated or covalently bonded to the hydrogel network.

### Example 10

### Use of Polyamidoamine Dendrimer Hydrogel for Ocular Drug Delivery: In Vitro And Ex Vivo Evaluation of Brimonidine and Timolol Maleate Dendrimer Hydrogel Formulations

### Introduction

Polyamidoamine (PAMAM) dendritic hydrogel (DH), constituted of dendritic nanoparticles crosslinked with polyethylene glycol (PEG), uniquely integrates the characteristics of highly branched dendrimers with a PEG network. For ocular drug delivery, DH promises to have properties superior to dendrimer or PEG gel alone, each of which has proven to be efficient as ophthalmic vehicles. Therefore, the objective of the work described in this Example was to demonstrate the feasibility of utilizing DH to fabricate a topical formulation for ocular drug delivery. The antiglaucoma drugs brimonidine and timolol maleate were used as model drugs and are representative of a variety of drugs that may be delivered to the eye of a patient. Cytotoxicity of DH formulations and their ability to enhance water solubility of hydrophobic brimonidine were studied. Further, in vitro drug release, cellular uptake, and ex vivo transcorneal transport and eye tissue uptake of the two drugs mediated with DH formulations were examined.

Previously, PEG chains were conjugated to amine-terminated PAMAM dendrimer first, and then photoreactive acrylates were introduced to the dendrimer. In the present Example, PEG chains were acrylated first, before reaction with the PAMAM dendrimer. PEG diol (Mn=12000 g·mol⁻¹) (1 eq.) dissolved in tetrahydrofuran (THF) was modified with acryloyl chloride (1 eq.) in the presence of triethylamine (TEA) (1 eq.). After overnight reaction, the salt was removed by centrifugation. To the supernatant 4-nitrophenyl chloroformate (NPC) (1 eq.) and TEA (1 eq.) were added. The reaction proceeded overnight while stirring. Upon the centrifugal removal of the salt, the resultant NPC-PEG-acrylate was dried through rotary evaporation. NPC-PEG-acrylate was then coupled to PAMAM dendrimer G3.0 in dimethylformamide. After 24-h reaction, G3.0-PEG-acrylate conjugates were then precipitated in cold ether, dialyzed against dionized water, and freeze-dried. The conjugates were characterized with ¹H-NMR spectroscopy. G3.0 coupled with an average of 3 PEG acrylate chains was obtained and used to prepare antiglaucoma drug formulations.

### Preparation of Antiglaucoma Drug Formulations

Single drug DH formulations (i.e., brimonidine 0.1% w/v and timolol maleate 0.5% w/v) and codrug DH formulations were prepared by suspending appropriate amounts of brimonidine, timolol maleate, or both in G3.0-PEG-acrylate solution (8.1% w/v in PBS) and then mixing the solution with eosin Y photoinitiator solution at a ratio of 5:100 v/v. Plain DH formulations (no drug content) were prepared by mixing G3.0-PEG-acrylate PBS solution (8.1% w/v) and eosin Y photoinitiator solution at a ratio of 5:100 v/v. The eosin Y photoinitiator solution contained eosin Y (0.1 wt %), TEOA (40 wt %), and 1-vinyl-2 pyrrolidinone (NVP) (4 wt %). All DH formulations were exposed to long-wave (365 nm) UV light for 30 min and kept overnight under ambient light prior to use. For comparison, single drug and codrug eye drop formulations were prepared by suspending brimonidine (0.1% w/v), timolol maleate (0.5% w/v), or both in PBS.

### LC-MS/MS Analysis

The concentration of brimonidine and timolol in study samples were measured by means of LC-MS/MS. An API-3000 triple quadrupole mass spectrometry (Applied Biosystems, Foster City, Calif., USA) coupled with a PerkinElmer series-200 liquid chromatography (Perkin Elmer, Walthm, Mass., USA) system was used. Analytes were separated on Zorbax extended C18 column (2.1×50 mm, 5 μm) using 5 mM ammonium formate in water (A) and acetonitrile (B) as mobile phase. The linear gradient elution at a flow rate of 0.3 ml/min with total run time of 6 mM was as follows: 60% A (0-1.0 min), 10% A (2.0→4.0 mM), and 60% A (4.5-6.0 min). Brimonidine, timolol and dorzolamide (internal standard) were analyzed in positive ionization mode with the following multiple-reaction monitoring (MRM) transitions: 292→212 (brimonidine); 317→261 (timolol); and 325→199 (dorzolamide).

### Statistical Analysis

Data were analyzed with analysis of variance (ANOVA) followed by t-test for pairwise comparison of subgroups using SigmaPlot 11.0 (Systat Software Inc., San Jose, Calif.). P values<0.05 were considered statistically significant.

### Enhancement of Water Solubility by Hydrogels

Experiments were carried out to determine whether the hydrogel of the invention is able to enhance water solubility of hydrophobic ocular drugs.

### Experimental

To estimate the degree of dissolution of hydrophobic brimonidine (Sigma-Aldrich, St. Louis, Mo.) in the presence of hydrogel G3.0-PEG-dA, an excess amount of brimonidine was added to 8.1% (w/v) G3.0-PEG-dA PBS solution and vortexed. Following overnight equilibration at room temperature, the solution was vortexed again and then centrifuged at 10,000 rpm for 5 min to remove undissolved drug. The sample solution was diluted by a factor of 100 in PBS, and absorbance value (Y) at 248 nm was recorded on a GENESYS™ 6 UV-Visual spectrophotometer. Thus, drug concentration (C in μg/mL) was determined using the following regression equation: C=(Y−0.005)/0.063. Following the same procedure, the solubility of brimonidine in plain PBS at room temperature was determined for comparison. Measurements were done in duplicate.

### Results

The saturated concentration of brimonidine in PBS at room temperature was 392.06 μg/ml, while the saturated concentration of briomonidine in the presence of G3.0-PEG-dA was 696.03 μg/ml, indicating 77.5% solubility increase.

### Cytotoxicity Assays Study of the Cytotoxicity of Four Hydrogel Formulations on Human Corneal Epithelial Cells Using MTT Assays

Four formulations were prepared based on dendrimer-PEG-acrylate with various loading degrees of PEGylation (A, 8:1; B, 6:1; C, 3:1; D, 1:1 as determined by ¹H-NMR) and tested to determine the formulation with minimum toxicity to cells. The sample preparation procedure can be found above.

### Experimental Design and Procedure

Human corneal epithelial cells (HCET, passage #40) were plated in 96-well plates at a seeding density of 5000 cells/well and allowed to adhere to the well for 24 hours. After 24 hours, cells were incubated with the four different formulations (30 μl each) for 24 hours. Three wells were kept as a control which contained only HCET cells.

The media was removed by aspiration after 24 hours and 100 μl of fresh serum free medium was added to each well at the end of 24 hours. MTT reagent (Sigma Aldrich, Mo.) i.e. 3-(4,5-dimethylthiazol-2-yl)-2,5-diphenyl tetrasodium bromide), (5 μl of 5 mg/ml MTT dissolved in PBS pH 7.4) was added to each well and incubated at 37° C. for 3 h. The medium was aspirated out and the formazan crystals formed were dissolved in 200 μl of DMSO. The absorbance of the color developed was measured at 570 nm using a microplate reader.

Micro BCA® Protein Assay Kit (Catalogue # 23235, Pierce Biotechnology, Inc. IL) was used to estimate the protein content of each well. BCA reagent MA, MB and MC were mixed in the ratio 50:48:2. Each of the well contents (150 μl) was added to 150 μl of the reagent mixture and kept at 37° C. for 2 hours. The elute reagent mixture absorbance was measured spectrophotometrically at 562 nm. A standard curve was made using bovine serum albumin (2 mg/ml stock solution, provided along with the kit) was used to make a standard curve for the protein estimation. Concentrations of 1.56, 3.125, 6.25, 12.5, 25, 50, 100, 500 μg/ml were made for the standard curve.

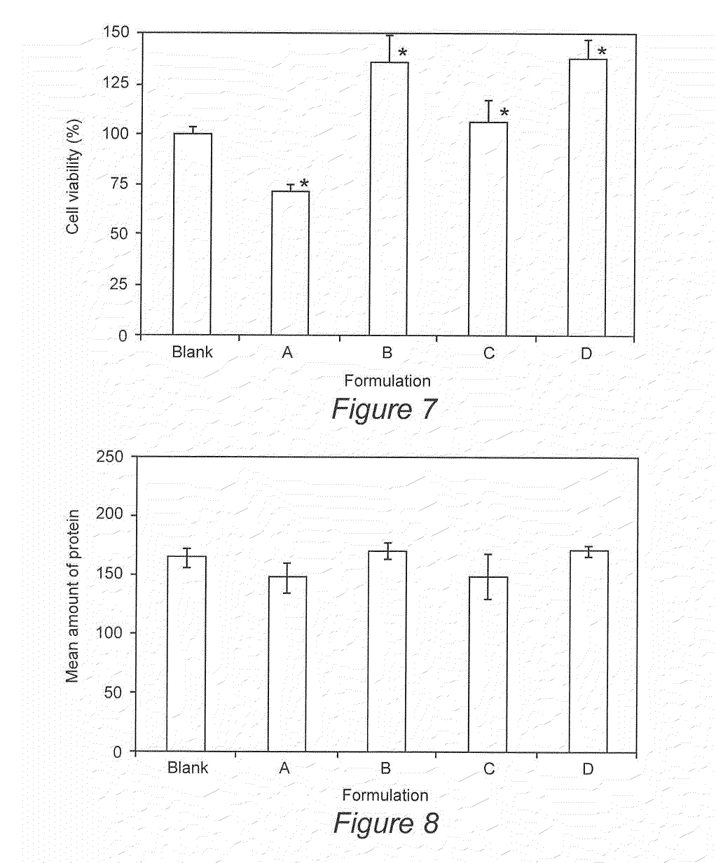

The results of the study are presented in FIG. 7. As can be seen, out of the 4 different formulations tested, formulation A was toxic to the cells and resulted in cell death whereas Formulations B and D were found to induce cell proliferation. However, Formulation C was not toxic to HCET cells and also did not induce cell proliferation. However, we wanted to confirm the effect by estimating the protein content in each well. Differences in protein content are used to confirm the effects (toxic or inducing proliferation) and aid in selecting the best formulation.

As can be seen in FIG. 8, the protein content estimation correlated well with the results of the MTT assays. Wells of Formulation A had less protein (734.5 μg/ml) than the blank (819.3 μg/ml). Similarly the increase in cell viability caused by Formulations B and D correlated with an increase in the protein content of the cells exposed to these formulations (852.6 and 853.2 for A and D respectively). Formulation C was found to be optimal and was selected to prepare formulations for further studies.

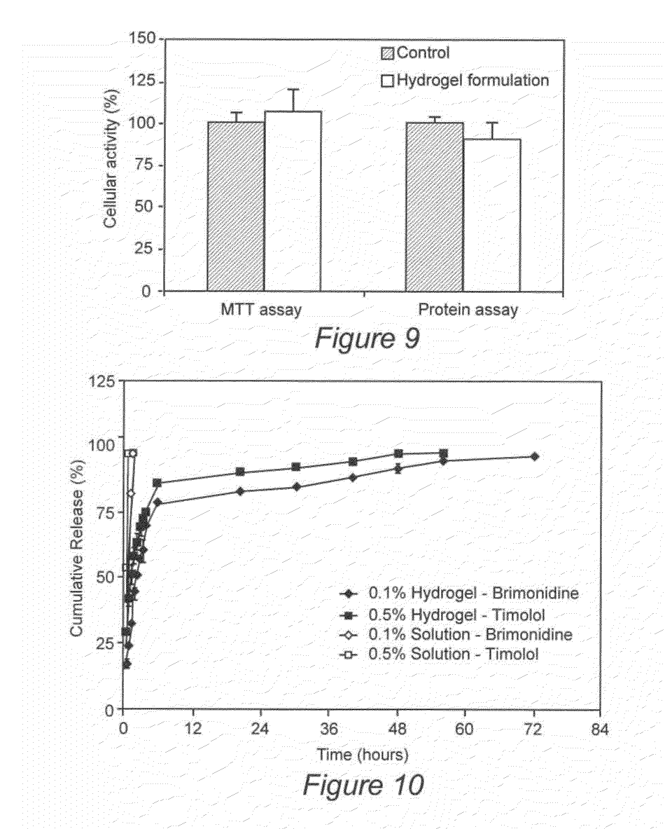

The MTT assay and protein assay results of Formulation C were normalized to control based on the data presented in FIG. 7 and FIG. 8 and presented in FIG. 9.

### Brimonidine Solubility Studies

To estimate the degree of dissolution of hydrophobic brimonidine (Sigma-Aldrich, St. Louis, Mo.) in the presence of G3.0-PEG-acrylate, an excess amount of brimonidine was added to G3.0-PEG-acrylate PBS solution (8.1% w/v) and vortexed. Following overnight equilibration at room temperature, the solution was vortexed again and then centrifuged to remove undissolved drug. The supernatant was collected, diluted by a factor of 100 in PBS, and its absorbance value (Y) at 248 nm was recorded on a GENESYS™ 6 UV-Visual spectrophotometer. Thus, drug concentration [C] (μg/mL) was determined using the following regression equation: C=(Y−0.005)/0.063 [2]. Following the same procedure, the solubility of brimonidine in plain PBS at room temperature was determined for comparison. Measurements were done in duplicate.

The results showed that the saturated concentration of brimonidine in PBS at room temperature was 392.06 μg/ml, while the saturated concentration of briomonidine in the presence of G3.0-PEG-dA was 696.03 μg/ml, indicating 77.5% solubility increase.

In Vitro Drug Release Studies: Study of Drug Release of Brimonidine and Timolol from Hydrogel Formulation C in pH 7.4 Phosphate Buffered Saline (PBS)

Hydrogel formulations entrap drug molecules in a matrix and are expected to release the drug at a sustained pace. The following drug release study at 37° C. helped to clarify the release profile, in comparison to that of a control eye drop formulation.

Hydrogel was formulated as described for Formulation C in Example 11. The formulation contained 0.1% brimondine and 0.5% timolol maleate and was evaluated for drug release as described below. The control eye drop formulation was PBS containing 0.1% brimondien and 0.5% timiolol maleate.

1. 100 μl of hydrogel formulation and 100 μl of eye drop formulation were transferred to separate 7 Spectra/Por® dialysis membrane bags (Spectrum Laboratories, Inc., CA; molecular weight cut off of 2,000 Da).
2. Each dialysis bag was suspended in 1.5 ml of dissolution media (pH 7.4 phosphate buffer containing 0.05% sodium azide).
3. The dissolution medium was maintained at 37±2° C. and constantly agitated in a shaker incubator.
4. Dissolution medium was completely replaced at all time intervals with 1.5 ml of fresh dissolution medium maintained at 37±2° C.
5. The amount of drug released in the dissolution medium at each time interval was analyzed by LC-MS.

FIG. 10 depicts the release profile. As can be seen, release of brimonidine and timolol was sustained from the hydrogel till 3 and 2½ days, respectively. Contrastingly, release from the eye drop formulation was immediate with the entire drug releasing within the first 1½ hours.

### In Vitro Drug Uptake Studies

The uptake of brimonidine and timolol maleate by HCET cells after entrapment of the drugs in a hydrogel was studied. As a control, eye drop formulation of both the drugs in phosphate buffer saline was used.
1. Human corneal epithelial cells (HCET, passage #40) were seeded in a 48 well plate (BD, Falcon®, Multiwell™ tissue culture plates) at a seeding density of 5000 cells/well.
2. The cells were allowed to adhere to the surface of the well overnight.
3. The next day the cells were exposed to 150 μl of hydrogel (n=4). 4 control wells were exposed to a suspension of brimonidine and a solution of timolol maleate in phosphate buffer saline (PBS) pH 7.4.
4. After an exposure of 1 hour, the formulations were removed from the wells and collected.
5. The cells were washed twice with 200 μl cold PBS pH 7.4 and twice with 200 μl cold acidic PBS pH 5.0. All the washes were collected. Finally, 200 μl of 1% w/v Triton X 100 solution was transferred to each well and allowed to stand for 30 minutes.
6. The cells were scraped (dislodged) using a pipette tip and suspension of cells in 1% Triton X 100 solution was collected.
7. Samples were processed as follows: 100 μl of sample collected as described above was diluted to 500 μl using acetonitrile. The samples were vortexed for 10 minutes and centrifuged at 10,000 rpm for 5 minutes, and then analyzed using LC-MS as described in Table 4. Dorzolamide was used as an internal standard

Tables 2 and Table 3 depict the drug content observed in the supernatants, washes and lysate of cells for hydrogel and eye drop formulations, respectively. As can be seen, in hydrogel formulation, brimonidine was found to be taken up by HCET cells up to 76.17% and timolol uptake was 69.1%. In comparison, the eye drop formulation showed an uptake of only 3.8 and 49.4% for brimonidine and timolol, respectively. Thus, a higher uptake by HCET cells was observed when brimonidine and timolol were entrapped in hydrogel formulation.

TABLE 2
Drug content (%) observed in cell uptake
study of hydrogel formulation (n = 3)

| | | |
|---|---|---|
| | Mean % drug | Standard |
| Drug content in | content | deviation |

Hydrogel formulation: Brimonidine

| | | |
|---|---|---|
| Supernatant after completion of study | 0.00 | 0.00 |
| First wash with acidic buffer | 0.00 | 0.00 |
| Second wash with acidic buffer | 0.00 | 0.00 |
| Cell lysate after completion of study | 76.17 | 19.47 |

Hydrogel formulation: Timolol

| | | |
|---|---|---|
| Supernatant after completion of study | 0.1 | 0.1 |
| First wash with acidic buffer | 0.0 | 0.0 |
| Second wash with acidic buffer | 14.9 | 2.79 |
| Cell lysate after completion of study | 69.1 | 13.7 |

TABLE 3
Drug content (%) observed in cell uptake
study (n = 3) of eye drop formulation

| | | |
|---|---|---|
| | Mean % drug | Standard |
| Drug content in | content | deviation |

Eye drop formulation: Brimonidine

| | | |
|---|---|---|
| Supernatant after completion of study | 0.06 | 0.04 |
| First wash with acidic buffer | 93.13 | 9.71 |
| Second wash with acidic buffer | 2.92 | 2.57 |
| Cell lysate after completion of study | 3.81 | 0.28 |

Eye drop formulation: Timolol

| | | |
|---|---|---|
| Supernatant after completion of study | 0.2 | 0.2 |
| First wash with acidic buffer | 0.0 | 0.0 |
| Second wash with acidic buffer | 34.7 | 3.35 |
| Cell lysate after completion of study | 49.4 | 2.6 |

Ex Vivo Transcorneal Transport Studies

Experiments were carried out to assess if there is any enhancement in the transcorneal transport of brimonidine and timolol from hydrogel formulation compared to the plain solution forms of these drugs.

### Experimental Design and Procedure

1. Corneas were isolated from freshly excised bovine eyes and mounted in Using chambers.
2. 50 μl of hydrogel formulation containing 0.1% brimonidine and 0.5% timolol diluted with assay buffer to 1.5 ml was used on the donor side (n=5). Note: Both drugs were present together in this hydrogel/solution cocktail.
3. For comparison purposes, 50 μl of plain solution containing 0.1% brimonidine and 0.5% timolol diluted with assay buffer to 1.5 ml was used on the donor side of control corneas (n=5). Therefore, amount of brimonidine and timolol on the donor side was 50 μg and 250 μg respectively for both experimental and control corneas.
4. 200 μl of sample was collected from the receiver side with fresh assay buffer replacement at the end of 1, 2, 3, 4 and 6 h.
5. At the end of 6 h, donor samples were collected and tissues were removed from the chambers for drug analysis. pH of hydrogel solution in the donor chamber (6 h)=7.0, pH of plain solution in the donor chamber (6 h)=6.83.
6. Samples were stored at −80° C. prior to analysis.
7. Cumulative percent transport was normalized to the amount of drug present at zero time point.

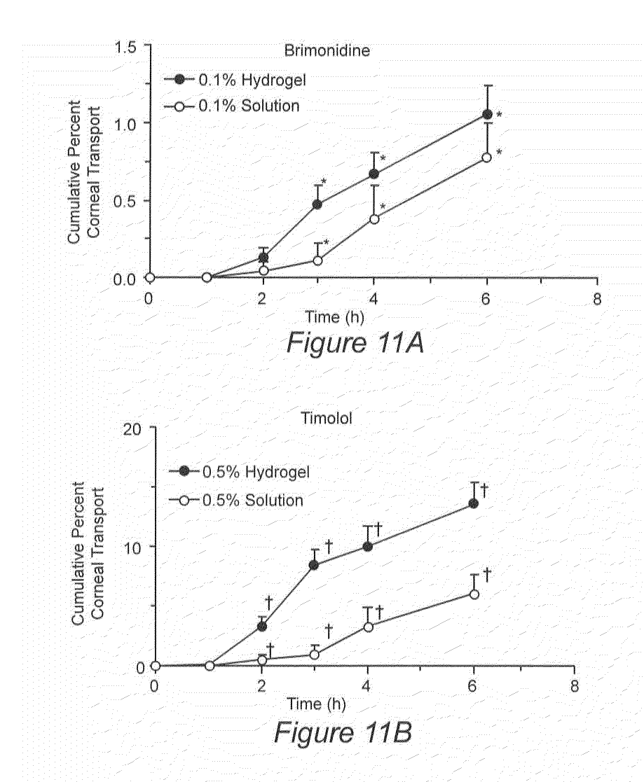

The results are depicted in FIGS. 11A and B. As can be seen, transport of timolol was found to be higher from both hydrogel and solution when compared with similar formulations of brimonidine. The reason may be attributed to the initial source amounts used (timolol amount was 5 times higher than brimonidine). At the end of 6 h, statistically significant differences were found in the corneal transport of timolol from hydrogel formulation and solution (p-value of 0.001) with timolol transport being higher from hydrogel. Similarly, statistically significant differences were found in the corneal transport of brimonidine from hydrogel formulation and solution (p-value of 0.05) where the hydrogel formulation showed slightly higher transport for brimonidine when compared with the solution.

### Ex Vivo Drug Uptake Studies

Uptake study of hydrogel formulation and solution containing 0.1% brimonidine and 0.5% timolol maleate into different bovine eye tissues after topical dosing

This study compared the uptake of brimonidine and timolol from hydrogel and solution dosage forms into different bovine eye tissues after topical dosing.

### Experimental Design and Procedure

1. Freshly excised bovine eyes were used for uptake study of hydrogel formulation (n=4) containing 0.1% brimonidine and 0.5% timolol and saline solution (n=4) containing 0.1% brimonidine and 0.5% timolol.
2. Eyes were kept in the muffin plate and partially dipped into PBS pH 7.4.
3. 50 μl of hydrogel formulation or solution was instilled gently as an eye drop onto the corneal surface.
4. After every 15 minutes, 50 μl of fresh PBS pH 7.4 was instilled as an eye drop to prevent corneal drying.
5. At the end of 1 h, eyes were dissected and the following tissues were collected: corneal epithelium, stroma, endothelium and aqueous humor. Tissues were stored at −80° C. before sample processing and analysis.

### Drug Extraction and Recovery

Extraction recovery of timolol and brimonidine from bovine corneal epithelium, stroma and endothelium:

1. Extraction recovery was done at 500 ng/ml for all of the above tissues. Standard solutions were analyte solution (25 μg/ml of timolol and brimonidine) and IS (dorzolamide, 25 μg/ml.
2. 20 mg of each tissue (epithelium, stroma, endothelium; n=5) was weighed in a glass tube. 20 μl of standard analyte solution (10 μl of IS and 470 μl of 2% NaOH solution, pH 12.8) was added to the above tube. Since both analytes as well as IS are highly basic molecules, a 2% NaOH solution was used to keep them in an un-ionized state.
3. Tissues were homogenized, followed by sonication for 10 minutes.
4. 4 ml of organic solvent mixture (ethyl acetate:dichloromethane=1:1) was added and samples were vortexed for 15 minutes followed by centrifugation at 3000 rpm for 15 minutes.
5. Organic layer was separated, evaporated under nitrogen and samples were reconstituted in 1 ml of acetonitrile-water mixture (75:25) for LCMS/MS analysis.

TABLE 4
Percentage extraction recoveries of brimonidine
and timolol maleate at 500 ng/ml from bovine corneal
epithelium, stroma and endothelium (n = 5).

| Tissue | Brimonidine | Timolol Maleate |
|---|---|---|
| Corneal Epithelium | 123 ± 21.7 | 103 ± 17.6 |
| Corneal Stroma | 118 ± 41.2 | 120 ± 10.1 |
| Corneal Endothelium | 112 ± 17.7 | 114 ± 26 |

### Drug Quantification in Ex Vivo Uptake Studies

Drug level estimation of timolol and brimonidine into bovine corneal epithelium, stroma and endothelium after 1 h of topical administration of their hydrogel and solution dosage forms:

1. Tissues weights were recorded and 0.49 ml of 2% NaOH solution was added to these tissues along with 10 μl of IS of 12.5 μg/ml.
2. Samples were homogenized, sonicated, and extracted with organic solvent. Final reconstitution was done in 0.5 ml of acetonitrile-water mixture (75:25).

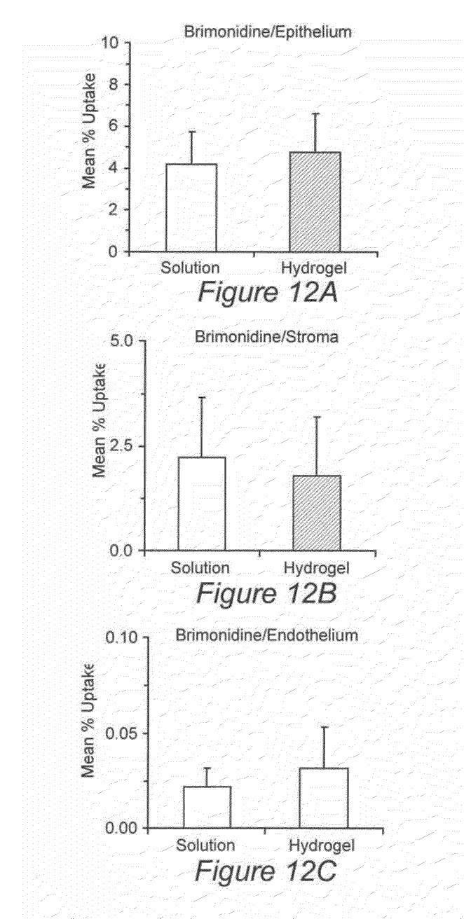

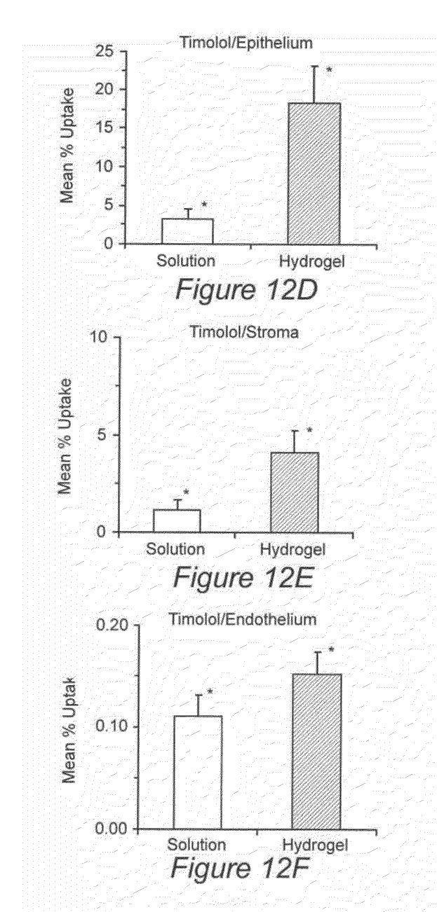

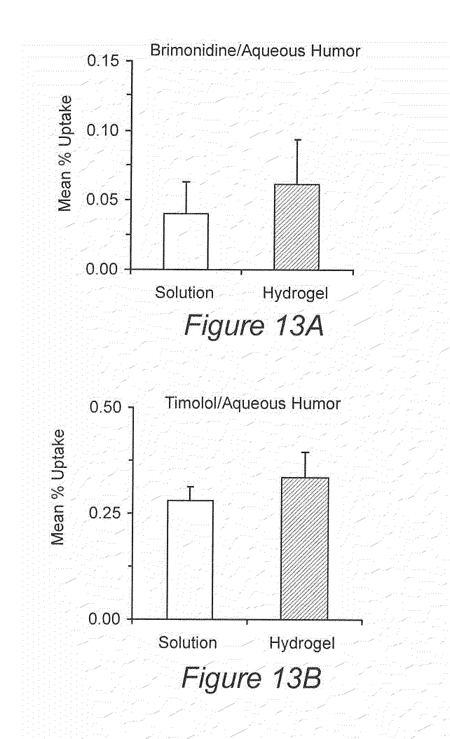

After one hour of instillation, significantly higher levels of both brimonidine (FIG. 12A-C), and timolol maleate (FIG. 12D-F) were observed in corneal epithelium, stroma and endothelium following hydrogel administration, compared to solution administration. However, this difference was not observed in aqueous humor levels at the end of one hour of uptake (FIGS. 13A and B). At the same time, corneal transport for timolol at the end of 6 h was significantly higher from hydrogel when compared with solution which might be attributed to the slow diffusion of the drug from the hydrogel over a long period of time (6 h).

### Results and Discussion

### Preparation and Toxicity Evaluation of Dendrimer Hydrogel (DH) Formulations

Recently, we have synthesized photocurable dendrimer derivatives, which are PAMAM dendrimers tethered with multiple polyethylene glycol (PEG) chains and photoreactive acrylate groups attached to the end of the conjugated polymer chains. Exposing these dendrimer-derivatives to suitable wavelengths of UV light triggers crosslinking of the reactive groups, leading to the formation of a dendrimer hydrogel (DH). DH integrates the characteristics and properties of both a dendrimer and PEG network. The surface charges conferred by terminal groups on the dendrimer surface can make the hydrogel polyionic with controllable charge density. The interior hydrophobic core of the dendrimer can encapsulate hydrophobic compounds, dramatically increasing their water solubility and loading amounts. Concurrently, the crosslinked PEG network can load hydrophilic drugs.

In our previous approach, described in Example 1, PEG chains were conjugated to amine-terminated PAMAM dendrimer first, and then photoreactive acrylates were introduced to the dendrimer by reacting acryloyl chloride with ideally the hydroxyl end groups of PEG chains. Acrylate attached to PEG would respond to UV light exposure to initiate crosslinking reaction. This approach has proven to be valid for gel formation. Due to the possible shielding effect of PEG, acrylate groups on the dendrimer surface should be avoided in order to achieve efficient crosslinking. However, restricting acrylate groups to the distal end of the conjugated PEG chains was beyond control in this approach as acryloyl chloride has reactivity towards free amine surface groups of the dendrimer. In this work, we modified this approach by reacting acryloyl chloride with PEG diol first to ensure that acrylate was restrictively attached to the end of PEG and then coupling PEG acrylate to the dendrimer. Photoreactive dendrimer derivatives in aqueous solutions are able to become viscous solutions and/or form “no flow” gels in situ upon light exposure by tuning their concentration and/or structure parameters including the degree of PEGylation, PEG length, and the density of acrylate groups on the dendrimer.

As a viscous gel solution is preferred to solid gel in ocular drug delivery due to its ease of handling and application, PAMAM dendrimer G3.0 coupled with an average of 3 PEG acrylate chains (i.e., Formulation C) was used to make viscous gel solutions for preparation of antiglaucoma-drug DH formulations. Unless specified, the DH formulations mentioned thereafter were based on Formulation C. The effect of plain DH formulations on cellular response was assessed. According to the MTT assay (FIG. 9), the DH formulation including photoiniator neither caused toxicity to HCET cells nor induced cell proliferation rate. The protein content in the cells was quantified by using the Micro BCA protein assay kit. It was shown that the protein content in the cells treated with the DH formulation was just 9.3% less than in the control (FIG. 9).

### Drug Water Solubility Enhancement

It has been documented that PAMAM dendrimers are able to increase the water solubility of hydrophobic compounds by encapsulating them inside the hydrophobic core. PEGylation of dendrimers can further augment such water solubility enhancement. As dendrimers have been integrated into a hydrogel network, one envisioned property of a dendrimer hydrogel is its ability to encapsulate hydrophobic drug molecules inside the dendritic cores, while simultaneously allowing the loading of hydrophilic drugs in the PEG network. To test the ability of the dendrimer hydrogel to enhance water solubility of hydrophobic drugs, brimonidine was used in this work. Unlike the commonly used water-soluble brimonidine tartrate in ophthalmic solutions, brimonidine has a limited solubility in aqueous solutions. Our studies revealed that the solubility of brimonidine was 392 μg/ml in plain PBS. In sharp contrast, the solubility of brimonidine dramatically increased to 696 μg/ml in DH formulation, representing a 77.6% increase.

### In Vitro Drug Release Studies

In vitro release of brimonidine and timolol maleate from DH formulation in pH 7.4 PBS was investigated. It was observed that drug release was sustained for nearly 72 h for brimonidine and nearly 56 h for timolol maleate (FIG. 10). Contrastingly, drug in eye drop formulations was released quickly. Both brimonidine and timolol maleate were released completely from eye drop formulations within one hour and a half, indicating the eye drop formulations did not sustain the drug release. Sustained drug release from DH formulations was attributed to the entrapment of drug molecules in the PEG network and the encapsulation by the nanodomains inside the dendrimers.

### Enhanced Drug Uptake

The intracellular uptake of brimonidine and timolol maleate by HCETs was substantially increased by the DH formulations. Table 2 and Table 3 summarize the drug content in the supernatants, acid washes and lysate of cells treated with DH and eye drop formulations, respectively. The eye drop formulations facilitated an uptake of only 3.8110.28% for brimonidine and 49.4012.60% for timolol maleate, respectively. With the aid of DH formulations, brimonidine uptake was 76.17119.47% and timolol uptake was 69.10113.70%. Particularly, the uptake of hydrophobic briomonidine mediated with the DH formulation was 19-fold higher than its uptake mediated with the eye drop formulation. Such dramatic cellular uptake of brimonidine was attributed to its increased water solubility and more even dispersion in the gel solution.

### Ex Vivo Transcorneal Transport

Transcorneal transport of brimonidine and timolol maleate was enhanced by the DH formulation as compared to the eye drop formulation. It was observed that the DH formulation indeed aided antiglaucoma drugs to cross the cornea at a higher rate than the eye drop formulation (FIGS. 11A and B). For brimonidine, statistically significant differences (p<0.05) were observed in its transcorneal transport starting from 3 h. For timolol maleate, statistically significant differences (p<0.001) were observed in its transcorneal transport from 2 h. In addition, the cumulative percentage transport of timolol maleate was much higher than that of brimonidine. For instance, at 6 h, only 1.06±0.18% of brimonidine from the DH formulation was transported across the cornea, while 13.54±1.83% of timolol maleate from the DH formulation was transported across the cornea.

### Ex Vivo Eye Uptake

This study was conducted to assess the ex vivo uptake of brimonidine and timolol maleate in ocular tissues after 1 h of topical instillation. The levels of brimonidine from hydrogel formulation were similar to those from eye drop solution formulation in corneal epithelium, stroma, and endothelium (FIG. 12A-C). We observed significantly higher levels of timolol maleate from hydrogel formulation as compared to eye drop solution in corneal epithelium, stroma and endothelium (FIG. 12D-F). Particularly, the timolol maleate levels from hydrogel formulation were 4.6-fold higher in epithelium, 2.6-fold higher in stroma, and 40% more in endothelium. However, significant difference in drug level was not observed in the aqueous humor between hydrogel formulation and eye drop formulation for both brimonidine and timolol maleate (FIGS. 13A and B).

### Conclusions

Dendrimer hydrogel was investigated as a formulation for antiglaucoma drug delivery. DH formulations displayed good cytocompatibility and could dramatically enhance water solubility of hydrophobic antiglaucoma drugs such as brimonidine. Brimonidine and timolol maleate encapsulated into dendrimer hydrogel were released in a sustained manner. The intracellular uptake of brimonidine and timolol maleate by HCETs and their transport across the bovine corneal endothelium were substantially increased by DH formulations as opposed to eye drop solution formulations. According to ex vivo bovine eye studies, significantly higher levels of timolol maleate in corneal epithelium, stroma and endothelium resulted from the application of the gel formulation. The in vitro and ex vivo studies indicate that dendrimer hydrogel formulations are capable of enhancing delivery of antiglaucoma drugs and represent a novel platform to deliver drugs for treatment of ocular diseases such as glaucoma. As a consequence, reduced dosing frequency and sustained efficacy of ocular drugs are expected.

### REFERENCES FOR EXAMPLE 10

1. Desai P N, Yuan Q, Yang H. Synthesis and characterization of photocurable polyamidoamine dendrimer hydrogels as a versatile platform for tissue engineering and drug delivery. Biomacromolecules 2010; 11:666-73.
2. Bhagav P, Deshpande P, Pandey S, Chandran S. Development and validation of stability indicating UV spectrophotometric method for the estimation of brimonidine tartrate in pure form, formulations and preformulation studies. Der Pharmacia Lettre 2010; 2:106-22.
3. Kompella U B, Sundaram S, Raghava S, Escobar E R. Luteinizing hormone-releasing hormone agonist and transferrin functionalizations enhance nanoparticle delivery in a novel bovine ex vivo eye model. Mol V is 2006; 12:1185-98.

### Example 11

### Testing of Hydrogel in Combination with Nanoparticles for Drug Delivery

Brimonidine and timolol maleate nanoparticles and gel suspensions were prepared to study the effect of these drugs on the intraocular pressure (IOP) of rabbit eyes in vivo. After a single topical dose, the rabbits were monitored for IOP till day 7. A suspension of timolol maleate and brimonidine was prepared in phosphate buffer saline pH 7.4 was used as a control. The concentration of the formulations was timolol maleate, 3.5% w/v; and brimonidine, 0.7% w/v.

### Preparation of Formulations

Three different formulations were prepared. These were

1. Nanoparticle formulation: Nanoparticles loaded with timolol maleate and brimonidine dispersed in PAMAM-G3.0-PEG-Acrylate hydrogel;
2. Hydrogel formulations: Timolol maleate and brimonidine dispersed in PAMAM-G3.0-PEG-Acrylate hydrogel; and
3. PBS dispersion formulations: Timolol maleate and brimonidine dispersed in phosphate buffer saline pH 7.4.

### Preparation of Nanoparticle Formulation

Nanoparticles were prepared by the conventional oil/water/water (o/w/w) emulsion technique. Poly (lactide-co-glyolide) polymer (Boeringher Ingelheim Inc, MW 30,000-35,000, IV-0.32-0.44 dl/g, 503H) was used for preparing the nanoparticles.

1. Polymer (100 mg) was dissolved in 1 ml of dichloromethane.
2. Timolol maleate (40 mg) and brimonidine (20 mg) (Sigma Aldrich, Inc., MO) were dispersed in the polymer solution.
3. The polymer and drug dispersion was added to sonication to 10 ml of an aqueous 2% poly vinyl alcohol solution using a probe sonicator (Misonix Sonicator 3000). The duration of sonication was 1 minute at a power input of 10 W.
4. The primary emulsion formed was further transferred to 50 ml of an aqueous poly vinyl alcohol solution under sonication. The duration of sonication was 3 minutes at a power input of 30 W.
5. The above secondary emulsion was continuously stirred at room temperature for 3 hours.
6. The nanoparticles formed were centrifuged at 13000 rpm (20000 g) for 15 minutes. The supernatant was discarded and the pellet of nanoparticles was redispersed in 25 ml of distilled water.
7. The nanoparticles were again centrifuged for 15 minutes at 13000 rpm (20000 g), then washing with distilled water was repeated.
8. The final nanoparticle pellet attained was redispersed in 10 ml of distilled water and the dispersion was frozen at −80° C. The frozen dispersion was subjected to lyophilization (Labconco lyophilizer, Labconco Corporation).
9. The drug content of the nanoparticles was estimated using liquid chromatography-mass spectrometry.
10. The nanoparticles were further dispersed in a gel as follows. Dispersion was made of 41 mg of hydrogel material (PAMAM-G3.0-PEG-Acrylate prepared as described below, except no drug was added to the hydrogel), nanoparticles equivalent to 17.5 mg of timolol maleate and 3.5 mg of brimonidine and 25 μl of photoinitiator solution. This dispersion was exposed to 20 mW/cm² of long-wave (365 nm) UV light at close range for 30 minutes. The gel nanoparticle dispersion was left under a UV light overnight.

### Preparation of Hydrogel Formulation

The method for preparation of hydrogel was as follows.

1. A quantity of 41 mg of dry hydrogel material (PAMAM-G3.0-PEG-Acrylate) was weighed in a microcentrifuge tube.
2. 17.5 mg of timolol maleate was dissolved in 500 μl of phosphate buffer saline
3. The above timolol solution was added to the tube containing dry hydrogel and vortexed for 2 minutes.
4. 3.5 mg of brimonidine was added to the timolol hydrogel material solution and vortexed for 2 minutes.
5. 25 μl of the photoinitiator solution was added to the tube and vortexed for 2 minutes.
6. The tube was exposed to 20 mW/cm² of long-wave (365 nm) UV light at close range for 30 minutes. The gel was left under a UV light overnight.

### Preparation of PBS Dispersion Formulations

A suspension of timolol maleate and brimonidine was prepared in phosphate buffer saline pH 7.4 by dissolving 17.5 mg timolol maleate and dispersing 3.5 mg brimonidine in 500 μl of phosphate buffer saline pH 7.4.

### In Vivo Study

### Animal Preparation and General Procedure

Adult Dutch belted male rabbits (purchased from Mrytle rabbitry, TN), 1.5-2.0 kg, were used in this study. The rabbits are provided with free access to food and water in a temperature-controlled room (18-24° C.). All rabbits used in these experiments are normotensive and housed under proper conditions, at the animal facility in Research Complex 2 at the University of Colorado Denver. All the procedures are conducted following approval by the IACUC of University of Colorado Denver. Intraocular pressure (IOP) was measured using a TONO-PEN AVIA® applanation tonometer (Reichert, Inc., NY). IOP is measured ten times at each interval and the mean taken. Rabbits that show any sign of eye irritation will be excluded from the study.

### Experimental Design

The above mentioned three formulations were tested. Three rabbits were used for each formulation (n=3). Each formulation (30 μL) was instilled topically into the upper quadrant of one eye (right) and the eye manually blinked three times. The IOP was measured at a suitable time intervals (30 min before dosing; 30 min, 1.5 hr, 3 hr, 6 hr post-dosing; 1, 2, 3, 5, 6 and 7 days post-dosing. IOP was also measured in the undosed eye at all mentioned time intervals. All procedures were conducting in the procedure room within the animal facility. Change in IOP is expressed as IOP dosed eye: IOP control eye and is reported as the mean±SD.

### Results of In Vivo Study

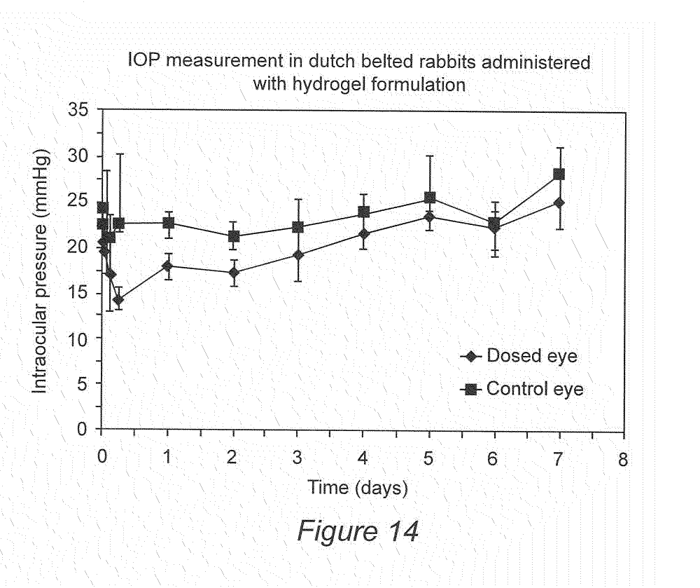

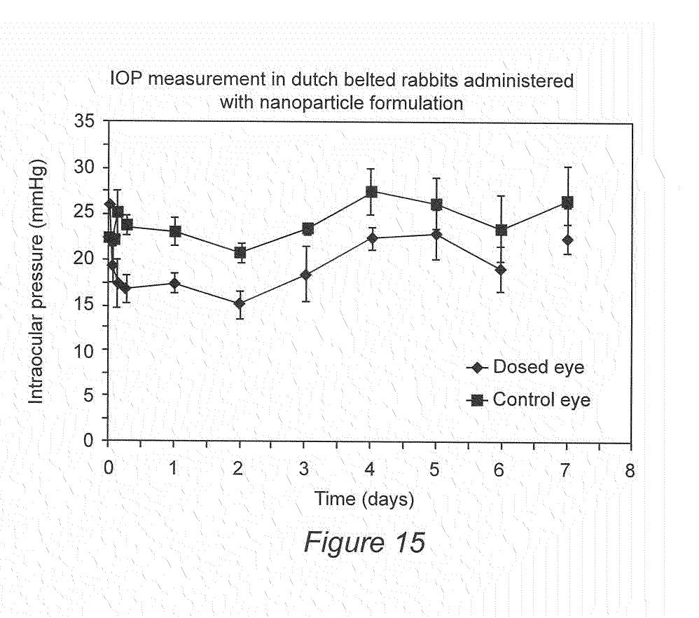

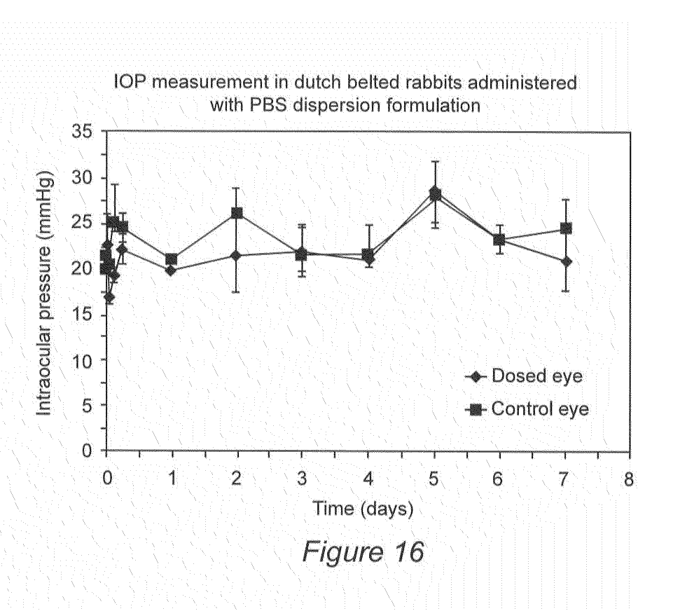

The results showed that dendrimer hydrogel formulation was able to achieve sustained efficacy over a period of 3 days (FIG. 14). Impressively, nanoparticle formulation (dendrimer hydrogel with encapsulated nanoparticles) was able to achieve sustained IOP control over a period of at least 7 days (FIG. 15). In contrast, the PBS formulation only resulted in IOP reduction for a few hours (FIG. 16).

### Example 12

### Human Corneal Cell Uptake of Nile Red Loaded Nanoparticles Entrapped in a Dendrimer Hydrogel Formulation

Studies were undertaken to investigate the uptake of nanoparticles entrapped in PAMAM-G3.0-PEG-Acrylate hydrogel by HCET (human corneal epithelial) cells. A suspension of Nile red loaded nanoparticles was prepared in phosphate buffer saline pH 7.4 and used as a control.

### Preparation of Formulations

Nanoparticles were prepared by the conventional o/w/w emulsion technique.

1. Poly (lactide-co-glyolide) polymer (Boeringher Ingelheim Inc, MW 30,000-35,000, IV-0.32-0.44 dl/g, 503H) was used for preparing the nanoparticles.
2. Polymer (100 mg) was dissolved in 1 ml of dichloromethane.
3. The polymer solution was added to 10 ml of an aqueous 2% poly vinyl alcohol solution using a probe sonicator (Misonix Sonicator 3000). The duration of sonication was 1 minute at a power input of 10 W.
4. The primary emulsion formed was further transferred to 50 ml of an aqueous poly vinyl alcohol solution under sonication. The duration of sonication was 3 minutes at a power input of 30 W.
5. The above secondary emulsion formed was continuously stirred at room temperature for 3 hours.
6. The nanoparticles formed were centrifuged at 13000 rpm (20000 g) for 15 minutes. The supernatant was discarded and the pellet of nanoparticles was redispersed in 25 ml of distilled water.
7. The nanoparticles were again centrifuged for 15 minutes at 13000 rpm (20000 g). Washing with distilled water was repeated again.
8. The final nanoparticle pellet attained was redispersed in 10 ml of distilled water and the dispersion frozen at −80° C. The frozen dispersion was subjected to lyophilization (Labconco lyophilizer, Labconco Corporation).
9. The nanoparticles were further dispersed in a gel as follows: A dispersion was made of 41 mg of hydrogel material (PAMAM-G3.0-PEG-Acrylate), 100 μg nanoparticles and 25 μl of photoinitiator solution. This dispersion was exposed to 20 mW/cm² of long-wave (365 nm) UV light at close range for 30 minutes. The gel nanoparticle dispersion was left under a UV light overnight.
10. 100 μg nanoparticles were dispersed in phosphate buffer saline pH 7.4 and used as a control.

### Cell Uptake Study

1. HCET cells were harvested from a T-75 flask at 80-90% confluency using trypsin-EDTA by adding 3 ml trypsin-EDTA to the dish, which was then incubated at 37° C. for 3-4 minutes.
2. Cells were lifted by tapping and the extent of detachment of cells was monitored under a microscope.
3. Once >90% of cells had detached, 5 ml of trypsin inhibitor and 5 ml of media were added to inhibit trypsin.
4. The media containing cells was transferred to a 15 ml conical tube.
5. The tube was centrifuged at 500 g for 5 minutes.
6. The supernatant was removed and the pelleted cells were resuspended in 5 ml of media.
7. 10 ul samples of the cell suspension were counted in a hematocytometer.
8. Cells were seeded in 48 well plates at a density of 80,000 cells/well.
9. Cells were allowed to adhere for 24 hours.
10. After 24 hours, 30 μl of a formulations containing 100 μg of nanoparticles was added to each well (n=6). To six control wells, 100 μg of nanoparticles dispersed in 30 μl of PBS pH 7.4 was added.
11. The cells were incubated with particles at 37° C. for 5 minutes, 60 minutes, or 3 hours.
12. At the end of 5 minutes, 60 minutes, or 3 hours, cells were washed twice with 0.25 ml acidic PBS (pH 5) and twice with 0.25 ml neutral PBS (pH 7.4) to remove particles sticking to the surface of the cells.
13. 0.25 ml PBS containing 2% triton-X 100 v/v was added to lyse the cells.
14. The cells were scraped and loosened with a pipette tip and collected into microcentrifuge tubes.
15. Fluorescence of the supernatant, washes, and cell lysate was measured in a spectrofluorometer plate reader at the excitation (485 nm) and emission (608 nm) wavelengths of Nile red.
16. Protein content was measured in the cell lysate using BCA assay kit.
17. Nanoparticle cell uptake was normalized with respect to protein content.
18. Standard curves were prepared for the nanoparticle suspension in pH 7.4 PBS buffer ranging from 1 mg/ml to 0.007 mg/ml. A standard curve was also prepared for the BCA protein estimation kit ranging from 1 mg/ml of bovine serum albumin to 0.07 mg/ml bovine serum albumin.

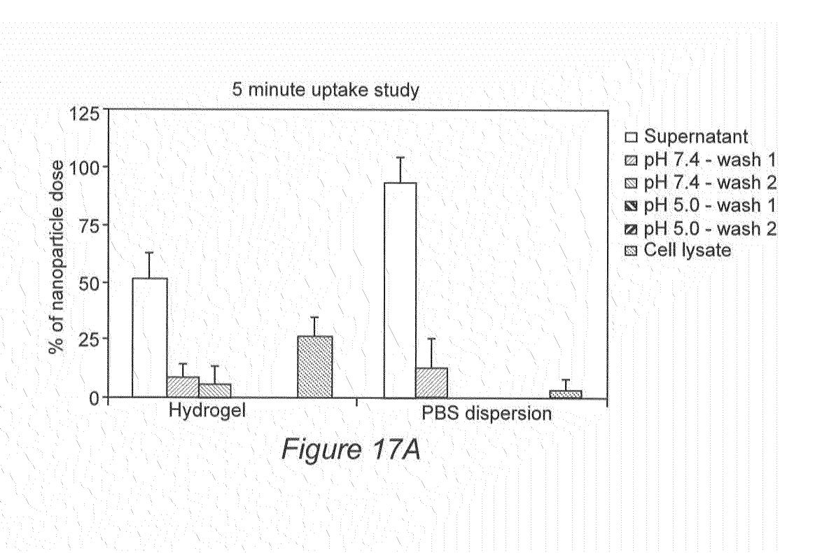

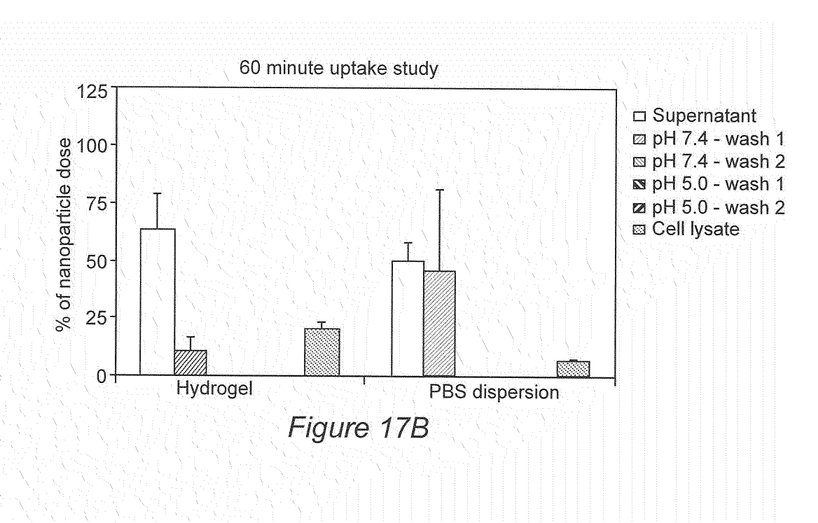

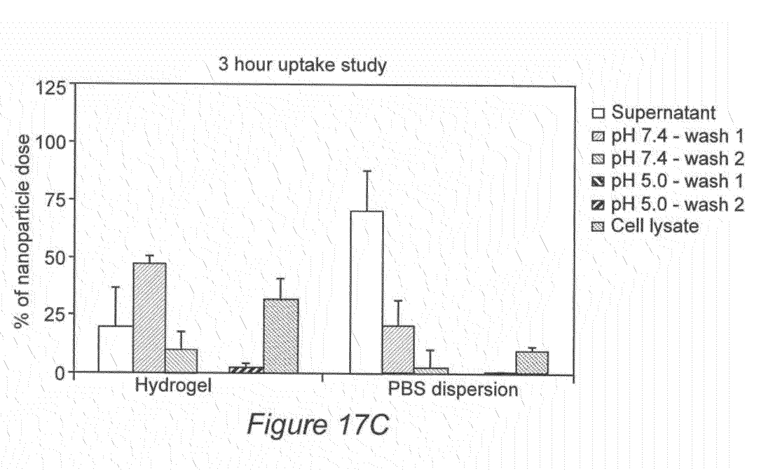

FIGS. 17A-C and Table 5 show the % of nanoparticle dose obtained in different cell solutions obtained after an incubation time of 5 minutes (FIG. 17A), 60 minutes (FIG. 17B), and 3 hours (FIG. 17C) with hydrogel or the PBS dispersion of nanoaprticles.

TABLE 5
Nanoparticle content in different solutions after incubation for 5 minutes, 60
minutes, and 3 hours. Data is shown as mean (±S.D.) for n = 6.

| | | % of nanoparticle dose | | | | | |
|---|---|---|---|---|---|---|---|
| | | 5 minutes | | 60 minutes | | 3 hours | |
| Hydrogel | supernatant | 51.66 | (±11.12) | 64.05 | (±16.43) | 20.26 | (±16.52) |
| | pH 7.4 wash 1 | 8.88 | (±5.95) | 11.86 | (±5.71) | 47.24 | (±2.82) |
| | pH 7.4 wash 2 | 6.07 | (±8.37) | 0 | (±6.82) | 9.64 | (±8.64) |
| | pH 5.0 wash 1 | 0 | (±0.0) | 0 | (±0.0) | 0 | (±0.92) |
| | pH 5.0 wash 2 | 0 | (±0.0) | 0.64 | (±0.61) | 2.58 | (±1.87) |
| | cell lysate | 26.39 | (±7.92) | 21.13 | (±3.25) | 31.5 | (±9.26) |
| PBS | supernatant | 94.42 | (±10.15) | 50.6 | (±8.42) | 70.14 | (±17.67) |
| Dispersion | pH 7.4 wash 1 | 13.39 | (±11.7) | 46.56 | (±35.56) | 21.01 | (±11.076) |
| | pH 7.4 wash 2 | 0 | (±0.0) | 0 | (±3.098) | 2.73 | (±7.65) |
| | pH 5.0 wash 1 | 0 | (±0.0) | 0 | (±0.0) | 0 | (±0.0) |
| | pH 5.0 wash 2 | 0 | (±0.0) | 0.24 | (±0.34) | 0.51 | (±0.33) |
| | cell lysate | 2.44 | (±3.73) | 7.95 | (±0.67) | 10.58 | (±1.64) |

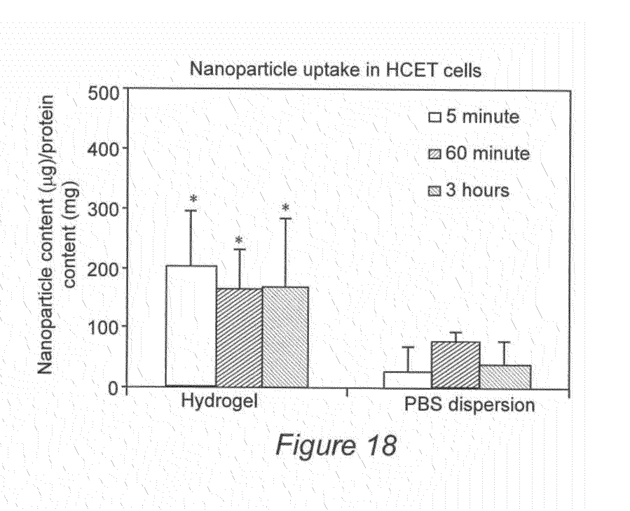

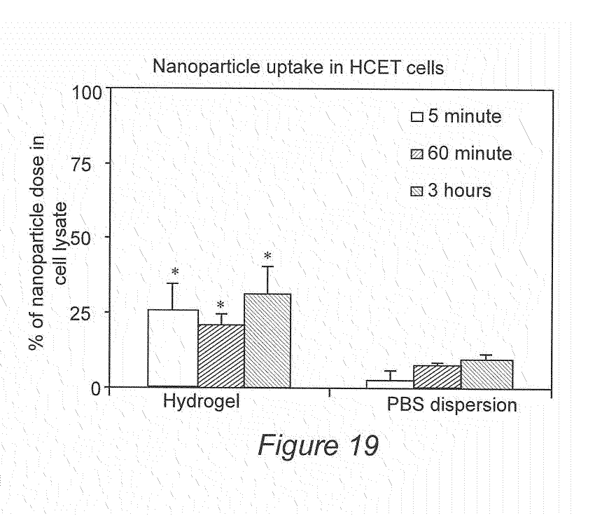

FIGS. 18 and 19 represent the nanoparticle content observed in the cell lysate only. This represents the amount of nanoparticles taken up by the cells after incubation with nanoaprticles entrapped in hydrogel or as PBS dispersion. FIG. 18 is the % of nanoparticle dose observed in cell lysate after different incubation times. FIG. 19 represents the data in μg nanoparticles normalized to the protein content observed in each well. T-test was used to calculate statistically significant differences in nanoparticle uptake by HCET cells.

### CONCLUSION

This example shows that nanoparticle uptake by cells was enhanced by entrapment in PAMAM-G3.0-PEG-acrylate hydrogel. The enhanced uptake was observed at all time points studied (5 minutes, 60 minutes, and 3 hours).

While the invention has been described in terms of its preferred embodiments, those skilled in the art will recognize that the invention can be practiced with modification within the spirit and scope of the appended claims. Accordingly, the present invention should not be limited to the embodiments as described above, but should further include all modifications and equivalents thereof within the spirit and scope of the description provided herein.

We claim:

1. A hydrogel-nanoparticle dispersion, comprising
i. a hydrogel comprising
a plurality of dendrimers, and
a plurality of crosslinked conjugated polymer chains; and
ii. nanoparticles dispersed in said hydrogel.

2. The hydrogel-nanoparticle dispersion of claim 1, wherein said crosslinked conjugated polymer chains are crosslinked at their termini.

3. The hydrogel-nanoparticle dispersion of claim 1, wherein said dendrimers are polyamidoamine (PAMAM) dendrimers.

4. The hydrogel-nanoparticle dispersion of claim 3, wherein said PAMAM dendrimers are PAMAM G3.0 dendrimers.

5. The hydrogel-nanoparticle dispersion of claim 1, wherein said conjugated polymer chains are polyethylene glycol (PEG) chains.

6. The hydrogel-nanoparticle dispersion of claim 5, wherein said PEG chains have a molecular weight of 12,000 Da.

7. The hydrogel-nanoparticle dispersion of claim 1, wherein said nanoparticles are foamed from copolymers of lactic acid and glycolic acid (PLGA).

8. The hydrogel-nanoparticle dispersion of claim 7, wherein said PLGA has a molecular weight of 2,000 to 100,000 Da.

9. The hydrogel-nanoparticle dispersion of claim 8, wherein said PLGA has a molecular weight of 30,000 to 35,000 Da.

10. The hydrogel-nanoparticle dispersion of claim 7, wherein a mass ratio of said PLGA to said hydrogel is 1:16.2.

11. The hydrogel-nanoparticle dispersion of claim 1, wherein said nanoparticles comprise at least one medicament.

12. The hydrogel-nanoparticle dispersion of claim 11, wherein said at least one medicament is a drug for treating a disease of the eye.

13. The hydrogel-nanoparticle dispersion of claim 12, wherein said disease of the eye is glaucoma and said at least one medicament includes one or both of timolol and brimonidine or salts thereof.

14. The hydrogel-nanoparticle dispersion of claim 13, wherein said salt of timolol is timolol maleate.

15. The hydrogel-nanoparticle dispersion of claim 13, wherein said at least one medicament includes 3.5% weight of timolol maleate per volume of hydrogel-nanoparticle dispersion and 0.7% weight of brimonidine per volume of hydrogel-nanoparticle dispersion.

16. The hydrogel-nanoparticle dispersion of claim 13, wherein said nanoparticles are formed from PLGA and wherein a weight ratio of timolol maleate to PLGA is 40:100 and a weight ratio of brimonidine to PLGA is 20:100.

17. A method for treating glaucoma in an eye of a subject, comprising the step of
administering to said eye of said subject a hydrogel-nanoparticle dispersion, comprising
i. a hydrogel comprising
a plurality of dendrimers, and
a plurality of crosslinked conjugated polymer chains; and
ii. nanoparticles dispersed in said hydrogel;
wherein said nanoparticles I include at least one medicament for treating glaucoma.

18. The method of claim 17, wherein said at least one medicament for treating glaucoma includes one or both of timolol and brimonidine, or salts thereof.

19. The method of claim 17, wherein said crosslinked conjugated polymer chains are crosslinked at their termini.

20. The method of claim 17, wherein said dendrimers are polyamidoamine (PAMAM) dendrimers.

21. The method of claim 20, wherein said PAMAM dendrimers are PAMAM G3.0 dendrimers.

22. The method of claim 17, wherein said conjugated polymer chains are polyethylene glycol (PEG) chains.

23. The method of claim 22, wherein said PEG chains have a molecular weight of 12,000 Da.

24. The method of claim 17, wherein said nanoparticles are formed from copolymers of lactic acid and glycolic acid (PLGA).

25. The method of claim 24, wherein said PLGA has a molecular weight of 2,000 to 100,000 Da.

26. The method of claim 25, wherein said PLGA has a molecular weight of 30,000 to 35,000 Da.

27. The method of claim 24, wherein a mass ratio of said PLGA to said hydrogel is 1:16.2.

28. The method of claim 18, wherein said timolol is timolol maleate and is present at 3.5% weight per volume of hydrogel-nanoparticle dispersion and said brimonidine is present at 0.7% weight per volume of hydrogel-nanoparticle dispersion.

29. The method of claim 18, wherein said timolol is timolol maleate and a weight ratio of said timolol maleate to PLGA is 40:100 and a weight ratio of brimonidine to PLGA is 20:100.

30. The method of claim 18, wherein said hydrogel-nanoparticle dispersion provides sustained release of said timolol and said brimonidine over a period of time in the range of from at least 1 to 7 days.

31. The method of claim 30, wherein said period of time is at least 7 days.

32. A method for forming a dendrimer hydrogel, comprising the steps of
covalently attaching photoactivatable reactive groups to terminal diol moieties of a plurality of polyethylene glycol (PEG)-diol polymer chains, thereby forming photoactivatable PEG polymer chains;
attaching said photoactivatable PEG polymer chains to a plurality of dendrimers; and
exposing a plurality of dendrimers with attached photoactivatable PEG polymer chains to a wavelength of light that causes cross-linking between photoactivatable reactive groups of said photoactivatable PEG polymer chains, thereby linking said plurality of dendrimers to each other via crosslinked PEG polymer chains and forming a dendrimer hydrogel.

33. The method of claim 32, further comprising a step of dispersing nanoparticles within said dendrimer hydrogel.

34. A dendrimer hydrogel, comprising
a plurality of PAMAM dendrimers;
a plurality of crosslinked conjugated polyethylene glycol (PEG) polymer chains;
one or more hydrophobic agents of interest contained within cores of said PAMAM dendrimers; and
one or more hydrophilic agents of interest associated with said crosslinked conjugated polyethylene glycol (PEG) polymer chains.

35. A method of intraocular delivery of a hydrophobic medicament and a hydrophilic medicament to a targeted location of a patient in need thereof, comprising the step of
delivering to said targeted location a dendrimer hydrogel comprising
a plurality of PAMAM dendrimers;
a plurality of crosslinked conjugated polyethylene glycol (PEG) polymer chains;
one or more hydrophobic agents of interest contained within cores of said PAMAM dendrimers; and
one or more hydrophilic agents of interest associated with said crosslinked conjugated polyethylene glycol (PEG) polymer chains.

36. The method of claim 35, wherein said targeted location is an eye.
